(19) United States
(12) Patent Application Publication
(10) Pub. No.: US 2010/0092532 A1
(43) Pub. Date: Apr. 15, 2010

(54) Materials and Methods for Treating and Managing Angiogenesis-Mediated Diseases

(76) Inventors: Helen Marie Nugent, Needham, MA (US); Elazer R. Edelman, Brookline, MA (US); Robert M. Tjin Tham Sjin, North Billerica, MA (US); Yin Shan Ng, North Billerica, MA (US)

Correspondence Address: K&L Gates LLP, STATE STREET FINANCIAL CENTER, One Lincoln Street, BOSTON, MA, 02111-2950, US

(21) Appl. No.: 12/513,564

(22) PCT Filed: Nov. 7, 2007
(86) PCT No.: PCT/US07/23535

§ 371 Date: Jun. 12, 2009

Related U.S. Application Data
(60) Provisional application No. 60/857,458, filed on Nov. 7, 2006.
(60) Provisional application No. 60/875,626, filed on Dec. 19, 2006.
(60) Provisional application No. 60/923,836, filed on Apr. 17, 2007.
(60) Provisional application No. 60/967,029, filed on Aug. 30, 2007.

(51) Int. Cl. A61K 9/00; A61K 35/12; A61P 9/00

(52) U.S. Cl. 424/422; 424/93.7

(57) ABSTRACT

Disclosed herein are materials and methods suitable for treating sites of pathological angiogenesis and abnormal neovascularization. Sites of pathological angiogenesis or abnormal neovascularization can be treated by contacting a surface at or adjacent or in the vicinity of an area of pathological angiogenesis or abnormal neovascularization with an implantable material. The implantable material comprises a biocompatible matrix and cells and is in an amount effective to treat the affected site. The composition can be a flexible planar material or a flowable composition. Diseases susceptible to treatment with the present invention include, for example, macular degeneration, rheumatoid arthritis, psoriasis, psoriatic arthritis, systemic inflammatory diseases, and treatment of tumors by surgical resection, radiation therapy or chemotherapy.

### RELATED APPLICATION DATA

This non-provisional patent application claims the benefit under 35 U.S.C. Section 119(e) of provisional patent application U.S. Ser. No. 60/857,458, filed on Nov. 7, 2006; provisional patent application U.S. Ser. No. 60/875,626, filed on Dec. 19, 2006; provisional patent application U.S. Ser. No. 60/923,836, filed on Apr. 17, 2007; and provisional patent application U.S. Ser. No. 60/967,029, filed on Aug. 30, 2007; the entire content of each of the foregoing incorporated by reference herein.

### BACKGROUND OF THE INVENTION

Angiogenesis, the process of growing new blood vessels from pre-existing vasculature, is involved in the natural healing processes resulting from wounds and surgical interventions, including the resection of solid tumorous cancer.

Abnormal neovascularization, distinct from pathological angiogenesis, is involved in the emergence and perpetuation of a variety of acute and chronic disease states, including macular degeneration, rheumatoid arthritis, psoriasis, psoriatic arthritis and other arthritic conditions and systemic inflammatory diseases, as described below.

For example, the exudative or neovascular form of age-related macular degeneration, corneal neovascularization and other neovascular diseases of the eye such as proliferative diabetic retinopathy, retinopathy of prematurity, Steven's-Johnson syndrome, cicatricial pemphigoid, corneal allograft rejection and corneal injury from infection or trauma are characterized by the abnormal growth of neovessels, inappropriately regulated and leaky blood vessels, in response to a hypoxic environment and unregulated inflammation. Left untreated, these diseases are major causes of blindness in persons of all ages. In age-related macular degeneration, for example, abnormal neovascularization produces sub-retinal hemorrhage, accumulation of fluid beneath the photoreceptors within the fovea, and neural cell death in the outer retina. If left untreated, the abnormal neovascular process usually results in subfoveal scar formation and blindness.

Synovial neovascularization is considered to be an important early step in the pathogenesis and perpetuation of rheumatoid arthritis. Abnormal neovascularization is integral to the development of inflammatory pannus, and without it leukocyte ingress could not occur. Furthermore, formation of new blood vessels permits a supply of nutrients and oxygen to the augmented inflammatory cell mass and so contributes to the perpetuation of synovitis.

Rheumatoid arthritis is traditionally considered a chronic, inflammatory autoimmune disorder that causes the immune system to attack the joints. It is a disabling and painful inflammatory condition, which can lead to substantial loss of mobility due to pain and joint destruction. Rheumatoid arthritis is a systemic disease, often affecting extra-articular tissues throughout the body including the skin, blood vessels, heart, lungs, and muscles. About 60% of rheumatoid arthritis patients are unable to work ten years after the onset of the disease.

Neovascularization appears to be a first-order event in both psoriasis and psoriatic arthritis. Abnormalities in the vascular morphology of the nail-folds of psoriasis patients without nail disease have been observed, as well as an increase in the number of synovial membrane blood vessels in psoriatic arthritis joint tissue. Psoriatic arthritis, or arthropathic psoriasis, is a type of inflammatory arthritis. Although psoriasis and psoriatic arthritis are inter-related disorders, psoriatic arthritis is a distinct condition with its own epidemiological, clinical and genetic features. It affects about 5-7% of people suffering from the chronic skin condition psoriasis. As well as causing joint inflammation, psoriatic arthritis can cause tendonitis and dactilytis. In addition, more than 80% of patients with psoriatic arthritis will have psoriatic nail lesions characterized by pitting of the nails, or more extremely, loss of the nail itself. Psoriatic arthritis can develop at any age. However, on average it tends to appear about 10 years after the first signs of psoriasis. For the majority of patients, this is between the ages of 30 and 50, but it can also affect children. Current treatment of psoriatic arthritis is similar to that of rheumatoid arthritis.

A significant angiogenesis-related disease is cancer. Cancer is the second leading cause of death in the United States and accounts for over one-fifth of all mortality. Although a variety of methods are known to treat cancer, including surgical resection of solid tumors, and radiation, ablation and chemotherapy to kill cancerous cells, local recurrence of cancerous cells and local damage or disease at the resected or treated site remain major challenges in the treatment of cancer patients. Surgical resection represents the greatest chance to treat localized disease that has not shown signs of metastasis to other locations in the body. Unfortunately, as many as 40% of cancer patients treated with surgical resection will develop a recurrence of the disease.

Current treatments for angiogenesis-related conditions and diseases and neovascularization-related conditions and diseases are limited. Treatment options vary with age, health, and the severity of the condition. One objective of the present invention is to provide methods and materials for the treatment of sites of abnormal neovascularization and pathological angiogenesis with the goal of locally directing healing of the affected or treated site.

### SUMMARY OF THE INVENTION

The present invention exploits the discovery that an implantable material comprising cells and a biocompatible matrix, when provided locally to a site of abnormal neovascularization or a site of pathological angiogenesis, can prevent, reduce or inhibit abnormal or pathological blood vessel formation, inflammation, extracellular matrix degradation and/or MMP expression and/or activation at the site to treat the affected or treated tissue or stroma and/or to prevent the growth or regrowth of pathological vasculature or other tissue at the affected or treated site. As disclosed herein, an implantable material comprising cells, preferably endothelial cells, cells having an endothelial-like phenotype, epithelial cells, cells having an epithelial-like phenotype, non-endothelial cells or analogs thereof can be used to prevent, heal, treat and manage sites of abnormal neovascularization or pathological angiogenesis when the material is situated on a surface of or within an affected or treated structure. This discovery permits the clinician to intervene in the treatment of a diseased tissue for which there have heretofore been limited treatment options.

One aspect of the present invention is a method of treating a site of pathological angiogenesis in an individual in need thereof, the method comprising contacting with an implantable material a surface at or adjacent to or in the vicinity of an area of angiogenesis, wherein said implantable material comprises a biocompatible matrix and cells and further wherein said implantable material is in an amount effective to treat the site of pathological angiogenesis in said individual.

According to additional embodiments, the biocompatible matrix is a flexible planar material or a flowable composition. The cells can be endothelial cells, endothelial-like cells, epithelial cells, epithelial-like cells or non-endothelial cells.

According to various embodiments, the implantable material regulates extracellular matrix degradation at the site of pathological angiogenesis, or regulates the expression and/or activation of MMPs at the site of pathological angiogenesis. According to one embodiment, the site of pathological angiogenesis is a site of tumor resection.

In another aspect, the invention is a composition suitable for the treatment or management of a site of abnormal neovascularization, the composition comprising a biocompatible matrix and cells, wherein said composition is in an amount effective to treat or manage the site of abnormal neovascularization.

According to additional embodiments, the biocompatible matrix is a flexible planar material or a flowable composition. The flowable composition can further comprise an attachment peptide and the cells are engrafted on or to the attachment peptide. The cells can be endothelial cells, endothelial-like cells, epithelial cells, epithelial-like cells or non-endothelial cells.

According to various embodiments, the composition regulates extracellular matrix degradation at the site of pathological angiogenesis or regulates expression and/or activation of MMPs at the site of pathological angiogenesis.

Another aspect of the present invention is a method of treating a site of abnormal neovascularization in an individual in need thereof, the method comprising contacting with an implantable material a surface at or adjacent to or in the vicinity of an area of abnormal neovascularization, wherein said implantable material comprises a biocompatible matrix and cells and further wherein said implantable material is in an amount effective to treat the site of abnormal neovascularization in said individual.

According to additional embodiments, the biocompatible matrix is a flexible planar material or a flowable composition. The cells can be endothelial cells, endothelial-like cells, epithelial cells, epithelial-like cells or non-endothelial cells.

According to various embodiments, the implantable material regulates extracellular matrix degradation at the site of abnormal neovascularization or regulates expression of MMPs at the site of abnormal neovascularization.

According to various embodiments, the site of abnormal neovascularization is a site of macular degeneration, a site of rheumatoid arthritis, a site of psoriasis, a site of psoriatic arthritis, or a site of another systemic inflammatory disease.

In another aspect, the invention is a composition suitable for the treatment or management of a site of abnormal neovascularization, the composition comprising a biocompatible matrix and cells, wherein said composition is in an amount effective to treat or manage the site of abnormal neovascularization.

According to additional embodiments, the biocompatible matrix is a flexible planar material or a flowable composition. The flowable composition can further comprise an attachment peptide and the cells are engrafted on or to the attachment peptide. The cells can be endothelial cells, endothelial-like cells, epithelial cells, epithelial-like cells or non-endothelial cells.

According to various embodiments, the composition regulates extracellular matrix degradation at the site of abnormal neovascularization or regulates expression and/or activation of MMPs at the site of abnormal neovascularization.

### BRIEF DESCRIPTION OF THE DRAWINGS

FIGS. 1A and 1B are representative cell growth curves according to an illustrative embodiment of the invention.

FIG. 2A depicts photographs of in vitro angiogenesis by HUVEC in Matrigel matrix with and without treatment with the implantable material of the present invention.

FIG. 2B is a graphical representation of the levels of angiogenesis or vascular tube density of HUVEC in Matrigel matrix following administration of conditioned media or the implantable material.

FIG. 3 is a graphical representation of the expression of MMP-2 in stained tissue sections of subjects treated with the implantable material and subjects administered the control material at 3 days and at 1 month.

FIG. 4 is a graphical representation of the expression of MMP-9 in stained tissue sections of subjects treated with the implantable material and subjects administered the control material at 3 days and at 1 month.

### DETAILED DESCRIPTION OF THE INVENTION

As explained herein, the invention is based on the discovery that a cell-based therapy can be used to treat, heal, ameliorate, manage and/or reduce the effects of pathological angiogenesis in conditions including sites of tumor resection. The cell-based therapy can also be used to treat, heal, ameliorate, manage and/or reduce the effects of abnormal neovascularization in conditions including macular degeneration, rheumatoid arthritis, psoriasis and psoriatic arthritis and other arthritic and systemic inflammatory conditions at the affected site and on surrounding tissues and stroma.

As used herein, the term angiogenesis means the process of forming or growing new blood vessels at a site of injury or disease as occurs during the normal healing process. As used herein, the term pathological angiogenesis means the process of forming abnormal and/or unwanted vessels at a site of injury or disease. Accordingly, the implantable material is preferrably administered prior to or at the time of disease onset, surgical procedure or intervention, or as a disruptive intervention to prevent and/or treat pathological angiogenesis.

As used herein, the term abnormal neovascularization means the result of pathological or abnormal emergence and growth of blood vessels as occurs at the onset of certain acute and chronic diseases and disorders. This is distinct from pathological angiogenesis. The present invention is useful to impair or prevent permanent establishment of abnormal ingrowth of blood vessels. It is contemplated that the composition of the present invention results in a localized environment which does not support an abnormal density of blood vessels. For example, even though blood vessels may grow into a particular site, the present invention limits or regulates those which ultimately reside permanently at a particular site. Accordingly, the implantable material is preferrably administered following emergence or onset of the disease or disorder, surgical procedure or intervention to treat abnormal neovascularization. However, the present invention can be used effectively as a disruptive intervention and/or to prevent a condition from worsening.

The teachings presented below provide sufficient guidance to make and use the materials and methods of the present invention, and further provide sufficient guidance to identify suitable criteria and subjects for testing, measuring, and monitoring the performance of the materials and methods of the present invention.

Macular Degeneration

Abnormal neovascularization occurs in a number of diseases of the eye including neovascular form of age-related macular degeneration, proliferative diabetic retinopathy, and retinopathy of prematurity. Left untreated, these diseases are major causes of blindness in infants (retinopathy of prematurity), working age adults (proliferative diabetic retinopathy) and the elderly (age-related macular degeneration). Neovascularizations, especially those that accompany the different diseases of the eye, are often induced in response to a hypoxic environment. However, the abnormal neovessels in these diseases are inappropriately regulated and leaky, and therefore abnormal neovessels contribute to the underlying pathology of the disease rather than correcting or compensating for the disease process.

In exudative or “wet” form of age-related macular degeneration, abnormal neovessels produce sub-retinal hemorrhage, accumulation of fluid beneath the photoreceptors within the fovea, and neural cell death in the outer retina. If left untreated, the abnormal neovascular process usually culminates in subfoveal scar formation. In premature infants suffering retinopathy of prematurity, the retinal vasculature is poorly developed and abnormal leaky neovessels grow into the avascular region of the retina. In severe and untreated cases, abnormal neovessels grow into the vitreous leading to retinal detachment and blindness. In diabetes, both direct effects of excessive glucose and indirect effects, possibly involving ischemia and hypoxia, on retinal tissue are believed to be responsible for the induction of abnormal neovascularization. Therefore, for each of these ocular pathologies, a logical treatment strategy is the regulation of pathological angiogenesis and the formation of abnormal neovessels using the implantable material of the present invention.

The implantable material of the present invention can be administered to an eye and/or surrounding tissues and stroma to treat, ameliorate, manage and/or reduce the clinical sequelae associated with age-related macular degeneration, proliferative diabetic retinopathy, retinopathy of prematurity, corneal neovascularization including Steven's-Johnson syndrome, cicatricial pemphigoid, corneal allograft rejection and corneal injury from infection or trauma and can inhibit or reduce the abnormal neovascularization at the site.

Rheumatoid Arthritis

Rheumatoid arthritis is a chronic systemic disease characterized by an inflammatory erosive synovitis. Early changes in the synovium are marked by abnormal neovascularization, inflammatory cell infiltration, and associated synoviocyte hyperplasia, which produce a pannus of inflammatory vascular tissue. This pannus covers and erodes articular cartilage, eventually leading to joint destruction. Abnormal neovascularization is recognized as a fundamental component of pannus development in rheumatoid arthritis, with evidence of both focal endothelial proliferation and apoptosis in the synovium. The implantable material of the present invention can be administered to a site of rheumatoid arthritis and/or surrounding tissues and stroma to treat, ameliorate, manage and/or reduce the clinical sequelae associated with rheumatoid arthritis and inhibit or reduce abnormal neovascularization at the site.

Psoriasis and Psoriatic Arthritis

Psoriatic arthritis, or arthropathic psoriasis, is a type of inflammatory arthritis. Although psoriasis and psoriatic arthritis are inter-related disorders characterized by neovascularization, psoriatic arthritis is a distinct condition with its own epidemiological, clinical and genetic features. It affects about 5-7% of people suffering from the chronic skin condition psoriasis. As well as causing joint inflammation, psoriatic arthritis can cause tendonitis and dactilytis. In addition, more than 80% of patients with psoriatic arthritis will have psoriatic nail lesions characterised by pitting of the nails, or more extremely, loss of the nail itself. Abnormalities in the vascular morphology of the nail-folds of psoriasis patients without nail disease have been observed, as well as an increase in the number of synovial membrane blood vessels in psoriatic arthritis joint tissue.

The implantable material of the present invention can be administered to a site of psoriasis or psoriatic arthritis and/or surrounding tissues and stroma to treat, ameliorate, manage and/or reduce the clinical sequelae associated with psoriasis and psoriatic arthritis and inhibit or reduce abnormal neovascularization at the site.

Systemic Inflammatory Conditions

In addition to rheumatoid arthritis, psoriasis and psoriatic arthritis, systemic inflammatory or autoimmune conditions also include, for example, systemic sclerosis (scleroderma), systemic lupus erythematosus, polymyositis/dermatomyositis, Sjören's syndrome, mixed connective tissue disease and systemic vasculitides. In these systemic inflammatory diseases, leukocytes emigrate into the diseased tissues through the vasculature. Increased abnormal neovascularization found in these inflammatory diseases further perpetuates the extravasation of leukocytes into the tissues, which further progresses the inflammatory disease.

Pathological angiogenesis and the outcome of diseases characterized by abnormal neovascularization, including systemic inflammatory or autoimmune conditions, are dependent on the balance or imbalance between neovascular mediators and inhibitors. In rheumatoid arthritis, for example, there is an excess of neovascular stimulators over neovascular inhibitors. In order to reset this balance, abnormal neovascularization needs to be suppressed, for example, by administering the implantable material of the present invention. The implantable material of the present invention can be administered to a site of abnormal neovascularization and/or surrounding tissues and stroma to treat, ameliorate, manage and/or reduce the clinical sequelae associated with the pathological angiogenesis, systemic inflammatory or autoimmune disease and inhibit or reduce the abnormal neovascularization at or near the site.

Tumor Resection

Tumor resection is the surgical removal of a solid tumor from a patient. Depending on the size of the tumor and its location, according to various embodiments, tumor resection can be performed in an open field surgical procedure or in a minimally-invasive surgical procedure, for example, a laproscopic surgical procedure. According to additional embodiments, tumor treatment procedures include radiation therapy, heat or light ablation therapy or chemotherapy to kill the tumor cells. For purposes of the present invention, it is contemplated that a resection site includes any and all sites subject to localized therapy for the treatment or removal of a tumor or tumor cell. Accordingly, resection sites include but are not limited to sites subject to surgical resection, radiation therapy, ablation therapy and/or chemotherapy.

In the case of surgical resection, following excision of a tumor, the tissues formerly surrounding the tumor create a pocket or resection site. In the case of radiation, ablation or chemotherapy, the tumor cells may remain transiently at the resection site. However, radiation, ablation and chemotherapy, in addition to killing the localized tumor cells, also damage the surrounding tissues and stroma. Accordingly, the resection site is prone to a variety of clinical sequelae, including, but not limited to, inflammation, extracellular matrix degradation, pathological angiogenesis and, in certain tumor types, regrowth of tumor cells at the resection margin and/or metastasis of the tumor cells.

Further, the tumor, and therefore the resection site, is often highly vascularized. Trauma to the resection site can result in pathological angiogenesis, extracellular matrix degradation, and other clinical sequelae associated with disruption of a resection site. The implantable material of the present invention can be administered to a resection site and/or surrounding tissues and stroma to treat, ameliorate, manage and/or reduce the clinical sequelae associated with trauma to the tumor resection site and inhibit or reduce pathological angiogenesis at the site. Pathological angiogenesis at the resected site is necessary for regrowth and/or metastasis of the tumor cells. Therefore, inhibition of pathological angiogenesis will inhibit and/or limit tumor re-growth or metastasis.

Additionally, the tumor microenvironment, including the stroma, is an important factor in controlling the re-growth and metastasis of tumors. The stroma comprises a variety of cells, including fibroblasts, myofibroblasts, glial cells, epithelial cells, immune cells including monocytes, macrophages, neutrophils and lymphocytes, vascular endothelial cells and smooth muscle cells along with the extracellular matrix and extracellular molecules. Often, due to their proximity to tumor cells and/or their interaction with each other, the cells of the stroma acquire an abnormal phenotype or altered function during or following contact with tumor cells. The implantable material of the present invention can be administered to a tumor stroma and/or surrounding tissues to treat, ameliorate, manage and/or reduce the clinical sequelae associated with disruption and/or an abnormal phenotype of the stroma.

Placement of the implantable material of the present invention at or adjacent to a site of surgical or clinical treatment is effective at diminishing mild and acute inflammation associated with the interventions as well as diminishing, delaying or inhibiting inflammation associated with the pathological angiogenesis condition. Furthermore, the implantable material also reduces MMP expression and/or activation, extracellular matrix degradation and pathological angiogenesis of affected or treated structures.

Matrix metalloproteinases (MMPs) are necessary for the migration of cells from the surrounding tissues into the injury site following injury by degrading extracellular matrix proteins. Activated myofibroblasts possess matrix degrading activities, which are regulated by the net balance between MMPs and their endogenous inhibitors, tissue inhibitors of matrix metalloproteinases (TIMPs). TIMPs are able to bind both to activated MMPs and to their inactivated precursors, pro-MMPs. See, Nagase, H., et al. “Matrix Metalloproteinases” J Biol Chem 274(31):21491-21494 (1999). The upregulation of MMPs and downregulation of TIMPs coincide with negative tissue remodeling following tissue injury. For example, adventitial expression of MMPs increases after vascular injury in AV graft models and facilitates the migration of fibroblasts to the neointima. See, Whatling, C. et al., “Matrix Management: Assigning Different Roles for MMP-2 and MMP-9 in Vascular Remodeling” Arterioscler. Thromb. Vasc. Biol. 24:10-11 (2004) and Galis, Z. S. et al., “Matrix Metalloproteinases in Vascular Remodeling and Atherogenesis: The Good, the Bad, and the Ugly” Circ. Res. 90:251-262 (2002).

The implantable material of the present invention can be administered to an affected or treated site, stroma and/or surrounding tissues to inhibit MMPs and/or to restore the proteolytic balance of MMPs and their inhibitors, TIMPs, to inhibit abnormal blood vessel growth and/or regrowth at the affected or treated site. Inhibition of abnormal blood vessel growth reduces the supply of nutrients available to nascent tumors or cancer stem cells and prevents the growth of tumors, metastasis of any remaining tumor cells and/or the maturation, differentiation and/or proliferation of cancer stem cells. Administration of the cell-containing implantable material of the present invention reduces MMP expression and pathological angiogenesis in affected and treated structures, compared to administration of a control material.

Pathological angiogenesis is necessary to support growth of tumor cells inadvertantly remaining following tumor resection. The implantable material of the present invention can be administered to a resection site, stroma and/or surrounding tissues to inhibit pathological angiogenesis at the resection site to therefore inhibit the supply of nutrients to a nascent tumor or cancer stem cell and prevent the growth of tumors, metastasis of any remaining tumor cells and/or the maturation, differentiation and/or proliferation of cancer stem cells.

Confluent endothelial cells release a variety of biological agents that in combination modulate MMP expression, angiogenesis and neovascularization. Endothelial cells seeded and allowed to proliferate in culture to confluence within a biocompatible matrix material can be implanted in a subject in need of treatment, producing the full gamut of endothelial inhibitory compounds. The implantable material of the present invention containing confluent endothelial cells can target multiple biological responses to injury. In contrast, administration of a single chemical agent can only respond to a single event. Endothelial cells within the implantable material secrete heparan sulfate proteoglycan, TGF-β, and TIMP-2 and while virtually all TIMPs form tight 1:1 inhibitory complexes with MMPs, they are also known to inhibit angiogenesis themselves. The endothelial cells within the implantable matrix also secrete nitric oxide (NO). Other studies have demonstrated that NO also decreases MMP activities and increases TIMP secretion in eNOS transfected rat smooth muscle cells. Decreased MMP activity and increased TIMP secretion correlates with inhibition of angiogenesis. Endothelial cells are able to deliver all endothelial derived compounds, including heparan sulfate, TGF-β, TIMP-2 and NO, in concert to decrease MMP expression and/or activation, extracellular matrix degradation, angiogenesis and neovascularization and to subsequently treat, heal and/or manage a site of pathological angiogenesis or abnormal neovascularization.

Accordingly, a cell-based therapy for clinically managing a site affected by pathological angiogenesis, abnormal neovascularization, and a condition and/or treatment to remove or reduce the condition, including managing inflammation, MMP expression and/or activation and extracellular matrix degradation has been developed. An exemplary embodiment of the present invention comprises a biocompatible matrix and cells suitable for use with the treatment paradigms described herein.

As used herein, the term “inhibition”, as applied to MMP activity, is intended to define a change in the level of biological activity of the MMP enzyme(s). Thus, modulation encompasses physiological changes which affect a decrease in MMP activity. The inhibition may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of MMP activity, or at the level of enzyme activity (for example by allosteric mechanisms, competitive inhibition, active-site inactivation, perturbation of feedback inhibitory pathways, etc.). Thus, MMP inhibition may imply suppressed expression or under-expression of the gene(s) encoding one or more MMP(s) at the transcriptional level, and/or decreased expression at the translational level. The terms “inhibited” and “inhibit” in relation to MMP activity are to be interpreted accordingly.

As used herein, the term “mediated”, as used in relation to MMPs or TIMPs in the context of any physiological process (e.g. tissue remodeling), disease, state, condition, therapy or treatment is intended to operate limitatively so that the various processes, diseases, states, conditions, therapies or treatments are those in which the MMPs or TIMPs play a biological role. The biological role played by the MMP or TIMP may be direct or indirect and may be necessary and/or sufficient for the manifestation of the symptoms of a disease, state or condition (or its aetiology or progression).

Implantable material of the present invention comprises cells engrafted on, in and/or within a biocompatible matrix. Engrafted means securedly attached via cell to cell and/or cell to matrix interactions such that the cells withstand the rigors of the preparatory manipulations disclosed herein. As explained elsewhere herein, an operative embodiment of implantable material comprises a near-confluent, confluent or post-confluent cell population having a preferred phenotype. It is understood that embodiments of implantable material likely shed cells during preparatory manipulations and/or that certain cells are not as securedly attached as are other cells. All that is required is that implantable material comprise cells that meet the functional or phenotypical criteria set forth herein.

The implantable material of the present invention was developed on the principals of tissue engineering and represents a novel approach to addressing the above-described clinical needs. The implantable material of the present invention is unique in that the viable cells engrafted on, in and/or within the biocompatible matrix are able to supply to the affected site multiple cell-based products in physiological proportions under physiological feed-back control. As described elsewhere herein, the cells suitable for use with the implantable material are endothelial, endothelial-like, epithelial, epithelial-like, non-endothelial cells or functional analogs of any of the foregoing cell types. Local delivery of multiple compounds by these cells and physiologically-dynamic dosing provide more effective regulation of the processes responsible for maintaining and healing affected sites, diminishing vascular supply to the sites and hence regrowth of pathological cells, pathological angiogenesis or reappearance of pathological indicia. The implantable material of the present invention, when contacted with an affected site, stroma or surrounding tissues, serves to reestablish homeostasis. That is, the implantable material of the present invention can provide an environment which mimics supportive physiology and is conducive to promoting and maintaining optimal levels of blood vessel density.

For purposes of the present invention, contacting means directly or indirectly interacting with an interior or exterior surface as defined elsewhere herein. In the case of certain preferred embodiments, actual physical contact is not required for effectiveness. In other embodiments, actual physical contact is preferred. All that is required to practice the present invention is deposition of an implantable material at, adjacent to or in the vicinity of an affected or treated site or stroma in an amount effective to treat the affected site or stroma.

For example, endothelial cells can release a wide variety of agents that in combination can inhibit or mitigate pathological angiogenesis, abnormal neovascularization, and adverse physiological events associated with acute complications following surgical or other treatment of pathological angiogenesis and abnormal neovascularization. As exemplified herein, a composition and method of use that recapitulates normal physiology and dosing is useful to enhance affected or treated structure stabilization. Typically, treatment includes placing the implantable material of the present invention at, adjacent to or in the vicinity of the affected or treated site, for example, within the eye, within an arthritic joint, at the surface of a psoriatic lesion, or in the interior space of the resection site created during the resection procedure in contact with the stroma. When deposited or otherwise contacting an affected or treated site, the cells of the implantable material can provide growth regulatory compounds to the site, for example to the underlying stromal cells within the site. It is contemplated that, when situated at an adjacent site, the cells of the implantable material provide a continuous supply of multiple regulatory compounds which can penetrate surrounding tissue and reach the site.

Treatment with a preferred embodiment of the present invention can encourage normal or near normal healing and normal physiology. On the contrary, in the absence of treatment with a preferred embodiment of the present invention, normal physiological healing is impaired, e.g., an increase in expression and activation of MMPs can result in adverse clinical consequences, such as inflammation, pathological angiogenesis, abnormal neovascularization, tumor regrowth and/or mestastasis. Accordingly, as contemplated herein, treatment with the implantable material of the present invention will improve the healing of native tissue at the treated site.

According to one embodiment, the implantable material is applied to an adjacent exterior surface or within the interior vitreous of an eye to support and promote healing of the macula at the affected site. Macular degeneration and surgical interventions to treat macular degeneration can disrupt the cells and tissues at and surrounding the macula and the eye and induce a cellular response, including, but not limited to, inflammation, abnormal neovascalarization and/or other clinical sequelae resulting from macular degeneration and surgical interventions to treat macular degeneration. Administration of the implantable material to the site of macular degeneration, stroma or surrounding tissue at the time of the treatment can reduce the incidence of clinical sequelae and encourage healing of the treated site.

According to another embodiment, the implantable material is applied to the interior cavity of an arthritic joint or to an adjacent exterior surface of the joint to support and promote healing of the affected ligament, tendon or capsule attachment to bone at the affected site. Rheumatoid and psoriatic arthritis and/or interventions to treat an arthritic structure can disrupt the cells and tissues surrounding the arthritic site and induce a cellular response, including, but not limited to, inflammation, abnormal neovascularization, extracellular matrix degradation and/or other clinical sequelae resulting from arthritis or surgical intervention. Administration of the implantable material to the arthritic site, stroma or surrounding tissue at the time of the treatment can reduce the incidence of clinical sequelae and encourage healing of the treated site.

According to a further embodiment, the implantable material is applied to the skin surface at or adjacent to a site of a psoriatic lesion to promote healing of the affected skin at the affected site. According to the embodiment, a bandage or other barrier is preferrably applied over the implantable material to maintain the effectiveness of the material during the course of treatment. Psoriasis can disrupt the epidermal cells and skin tissue layers at and adjacent to the psoriatic lesion and can induce a cellular response, including, but not limited to, inflammation, abnormal neovascularization, extracellular matrix degradation and/or other clinical sequelae. Administration of the implantable material to the psoriasis lesion site, stroma or surrounding tissue at the time of the treatment can reduce the incidence of clinical sequelae and encourage healing of the treated site.

According to another embodiment, the implantable material is applied to an affected or treated site following excision of a tumor to support the resection cavity and to promote healing of the tissue surrounding the resection site and at the resection margin. Surgical interventions to resect a tumor can disrupt the cells and tissues surrounding the tumor resection site and induce a cellular response, including, but not limited to, inflammation, pathological angiogenesis, extracellular matrix degradation and/or other clinical sequelae resulting from surgical resection. Administration of the implantable material to the resection site, stroma or surrounding tissue at the time of the resection, immediately following resection or at some later time following resection can reduce the incidence of clinical sequelae and encourage healing of the resected site.

According to one embodiment, the implantable material can promote and/or restore controlled proliferation and/or migration of vascular tissues. Macular degeneration, rheumatoid and psoriatic arthritis, psoriasis, and tumors recruit and/or promote pathological angiogenesis or abnormal neovascularization at the affected site to support uncontrolled cell growth. Accordingly, tumor sites and, therefore, resection sites, are highly vascularized tissues. Resection of a vascularized tumor necessarily results in trauma and/or damage to the vasculature supporting the excised tumor. Vascular trauma includes, but is not limited to, inflammation, thrombosis, stenosis and/or fibrosis of the disrupted vascular structures. Administration of the implantable material at the resection site contacting or adjacent to the disrupted vascular structures can reduce the incidence of vascular trauma associated clinical sequelae and inhibit pathological angiogenesis.

According to a further embodiment of the invention, the implantable material is applied to a resection site prior to removal of a tumor to isolate normal surrounding tissues from tumor tissue and/or to prevent the spread of disease to surrounding tissues and stroma or adjacent organs by inadvertant contamination by dislodged tumor cells during surgical excision. According to one embodiment, the implantable material is administered prior to excision of the tumor, for example, to prepare the surgical site for the resection procedure and/or to shield the surrounding tissues from possible tumor cell dislodgement during the resection procedure. According to another embodiment, the implantable material can be administered following resection of the tumor, for example, to reduce the movement of dislodged tumor cells from the vicinity of the resected tumor and to reduce the possibility of metastasis of the tumor cells.

According to various embodiments of the invention, the implantable material is applied to a variety of resected tumor sites, including, but not limited to, tumors of the bladder, bone, brain, breast, cervix, colon, esophagus, gallbladder, kidneys, larynx, liver, lung, mouth, ovaries, pancreas, prostate, stomach, testicles or thyroid.

For purposes of the present invention, it is believed that treatment with the implantable material of the present invention provides a beneficial homeostatic environment such that symptoms and complications common in systemic inflammatory and pathological angiogenesis conditions and related interventions, for example, inflammation, clotting and/or the growth of accessory veins, are reduced when the implantable material is placed adjacent to or in the vicinity of the affected or treated structure whether at the time of the intervention or at a later stage.

The implantable material of the present invention can be provided to the treated structure at any of a number of distinct stages. The implantable material can be provided during or after an initial surgical intervention to hasten healing generally, as well as to maintain the treated site in a clinically stable state. It is contemplated that the implantable material can be used not only at the time of initial treatment, but also at subsequent time points (e.g., for maintaining a treated site following the surgical procedure). Subsequent administrations can be accomplished surgically or non-invasively.

The materials and methods of the present invention can be used in connection with any of the above-described conditions, or numerous other treatment or management interventions. In addition, the materials and methods of the present invention can be used in connection with any intervention requiring surgery to improve surgical success and promote healing. The materials and methods of the present invention can be used in conjunction with these or other surgeries to increase effectiveness and promote healing.

Implantable Material

General Considerations: Implantable material of the present invention comprises cells engrafted on, in and/or within a biocompatible matrix. Engrafted means securedly attached via cell to cell and/or cell to matrix interactions such that the cells withstand the rigors of the preparatory manipulations disclosed herein. As explained elsewhere herein, an operative embodiment of implantable material comprises a near-confluent, confluent or post-confluent cell population having a preferred phenotype. It is understood that embodiments of implantable material likely shed cells during preparatory manipulations and/or that certain cells are not as securedly attached as are other cells. All that is required is that implantable material comprise cells that meet the functional or phenotypical criteria set forth herein.

The implantable material of the present invention was developed on the principals of tissue engineering and represents a novel approach to addressing the above-described clinical needs. The implantable material of the present invention is unique in that the viable cells engrafted on, in and/or within the biocompatible matrix are able to supply to the resected site multiple cell-based products in physiological proportions under physiological feed-back control. As described elsewhere herein, the cells suitable for use with the implantable material include endothelial, endothelial-like, epithelial, epithelial-like, non-endothelial cells or functional analogs of any of the foregoing cell types. Local delivery of multiple compounds by these cells in a physiologically-dynamic dosing provide more effective regulation of the processes responsible for reducing the symptoms associated with systemic inflammatory conditions, pathological angiogenesis and maintaining functional resected structures and diminishing the clinical sequelae associated with resection.

It is contemplated herein that a surface can be an exterior surface of an eye, a joint, the skin, or a resected structure, an interior surface of an eye, a joint, the skin or a resected structure, at a resection margin or within a tissue surrounding such structure. For purposes of this invention, surface is any component at or adjacent to of the affected or treated structure.

The implantable material of the present invention, when deposited or otherwise contacted with an affected or treated site or surrounding tissue serves to reestablish homeostasis. That is, the implantable material of the present invention can provide an environment which mimics supportive physiology and is conducive to treat or manage a site of treatment.

For purposes of the present invention, contacting means directly or indirectly interacting with an exterior or interior surface of an affected structure as defined elsewhere herein. In the case of certain preferred embodiments, actual physical contact is not required for effectiveness. In other embodiments, actual physical contact is preferred. All that is required to practice the present invention is exterior or interior deposition of an implantable material at, adjacent to or in the vicinity of an affected site in an amount effective to treat the site.

Endothelial cells can release a wide variety of agents that in combination can inhibit or mitigate adverse physiological events associated with acute and chronic complications due to, for example, a systemic inflammatory condition, pathological angiogenesis condition, abnormal neovascularization condition, or tumor resection. As exemplified herein, a composition and method of use that recapitulates normal physiology and dosing is useful to treat and manage such conditions. Typically, treatment includes placing the implantable material of the present invention at, adjacent to or in the vicinity of the affected site, for example, in the space previously occupied by the resected tumor or in direct contact with the stroma. When deposited at the affected site or otherwise contacting an affected site, the cells of the implantable material can provide regulatory compounds to the affected site. It is contemplated that, while contacting the affected site, the implantable material of the present invention comprising a biocompatible matrix or particle with engrafted cells provides a continuous supply of multiple regulatory compounds from the cells.

Cell Source: As described herein, the implantable material of the present invention comprises cells. Cells can be allogeneic, xenogeneic or autologous. In certain embodiments, a source of living cells can be derived from a suitable donor. In certain other embodiments, a source of cells can be derived from a cadaver or from a cell bank.

In one currently preferred embodiment, cells are endothelial cells. In a particularly preferred embodiment, such endothelial cells are obtained from vascular tissue, preferably but not limited to arterial tissue. As exemplified below, one type of vascular endothelial cell suitable for use is an aortic endothelial cell. Another type of vascular endothelial cell suitable for use is umbilical cord vein endothelial cells. And, another type of vascular endothelial cell suitable for use is coronary artery endothelial cells. Yet another type of vascular endothelial cell suitable for use is saphenous vein endothelial cells. Yet other types of vascular endothelial cells suitable for use with the present invention include pulmonary artery endothelial cells and iliac artery endothelial cells.

In another currently preferred embodiment, suitable endothelial cells can be obtained from non-vascular tissue. Non-vascular tissue can be derived from any anatomical structure, tissue or organ. Non-vascular tissue can be derived from any tissue type subject to treatment. Non-vascular anatomical structures include structures of the renal system, the reproductive system, the genitourinary system, the gastrointestinal system, the pulmonary system, the respiratory system and the ventricular system of the brain and spinal cord.

In yet another embodiment, endothelial cells can be derived from endothelial progenitor cells or stem cells. In still another embodiment, endothelial cells can be derived from progenitor cells or stem cells generally. In other preferred embodiments, cells can be non-endothelial cells that are allogeneic, xenogeneic or autologous and can be derived from vascular or non-vascular tissue or organ. Cells can be selected on the basis of their tissue source and/or their immunogenicity. Exemplary non-endothelial cells include epithelial cells, smooth muscle cells, fibroblasts, stem cells, endothelial progenitor cells, cardiomyocytes, secretory and ciliated cells. The present invention also contemplates any of the foregoing which are genetically altered, modified or engineered.

In a further embodiment, two or more types of cells are co-cultured to prepare the present composition. For example, a first cell can be introduced into the biocompatible implantable material and cultured until confluent. The first cell type can include, for example, secretory cells, smooth muscle cells, chondrocytes, fibroblasts, stem cells, endothelial progenitor cells, a combination of smooth muscle cells and fibroblasts, any other desired cell type or a combination of desired cell types suitable to create an environment conducive to endothelial cell growth. Once the first cell type has reached confluence, a second cell type is seeded on top of the first confluent cell type in, on or within the biocompatible matrix and cultured until both the first cell type and second cell type have reached confluence. The second cell type may include, for example, endothelial cells or any other desired cell type or combination of cell types. It is contemplated that the first and second cell types can be introduced step wise, or as a single mixture. It is also contemplated that cell density can be modified to alter the ratio of smooth muscle cells to endothelial cells.

To prevent over-proliferation of smooth muscle cells or another cell type prone to excessive proliferation, the culture procedure can be modified. For example, following confluence of the first cell type, the culture can be coated with an attachment factor suitable for the second cell type prior to introduction of the second cell type. Exemplary attachment factors include coating the culture with gelatin to improve attachment of endothelial cells. According to another embodiment, heparin can be added to the culture media during culture of the second cell type to reduce the proliferation of the first cell type and to optimize the desired first cell type to second cell type ratio. For example, after an initial growth of smooth muscle cells, heparin can be administered to control smooth muscle cell growth to achieve a greater ratio of endothelial cells to smooth muscle cells.

In a preferred embodiment, a co-culture is created by first seeding a biocompatible implantable material with smooth muscle cells to create structures, for example, but not limited to, structures that mimic the size and/or shape of the treatment site. Once the smooth muscle cells have reached confluence, endothelial cells are seeded on top of the cultured smooth muscle cells on the implantable material to create a simulated structure.

All that is required of the cells of the present composition is that they exhibit one or more preferred phenotypes or functional properties. As described earlier herein, the present invention is based on the discovery that a cell having a readily identifiable phenotype when associated with a preferred matrix (described elsewhere herein) can facilitate, restore and/or otherwise modulate cell physiology and/or cell or tissue homeostasis associated with the treatment of affected structures generally.

For purposes of the present invention, one such preferred, readily identifiable phenotype typical of cells of the present invention is an ability to inhibit or otherwise interfere with smooth muscle cell proliferation and/or migration as measured by the in vitro assays described below. This is referred to herein as the inhibitory phenotype.

One other readily identifiable phenotype exhibited by cells of the present composition is that they are anti-thrombotic or are able to inhibit platelet adhesion and aggregation. Anti-thrombotic activity can be determined using an in vitro heparan sulfate assay and/or an in vitro platelet aggregation assay, described below.

Another readily identifiable phenotype exhibited by cells of the present composition is the ability to restore the proteolytic balance, the MMP-TIMP balance, the ability to decrease expression of MMPs relative to the expression of TIMPs, or the ability to increase expression of TIMPs relative to the expression of MMPs. Proteolytic balance activity can be determined using an in vitro TIMP assay and/or an in vitro MMP assay described below.

A further readily identifiable phenotype exhibited by cells of the present composition is the ability to inhibit tube formation. Tube formation activity can be determined using an in vitro Matrigel assay described below.

In a typical operative embodiment of the present invention, cells need not exhibit more than one of the foregoing phenotypes. In certain embodiments, cells can exhibit more than one of the foregoing phenotypes.

While the foregoing phenotypes each typify a functional endothelial cell, such as but not limited to a vascular endothelial cell, a non-endothelial cell exhibiting such a phenotype(s) is considered endothelial-like for purposes of the present invention and thus suitable for use with the present invention. Cells that are endothelial-like are also referred to herein as functional analogs of endothelial cells; or functional mimics of endothelial cells. Thus, by way of example only, cells suitable for use with the materials and methods disclosed herein also include stem cells or progenitor cells that give rise to endothelial-like cells; cells that are non-endothelial cells in origin yet perform functionally like an endothelial cell using the parameters set forth herein; cells of any origin which are engineered or otherwise modified to have endothelial-like functionality using the parameters set forth herein.

Typically, cells of the present invention exhibit one or more of the aforementioned phenotypes when present in confluent, near confluent or post-confluent populations and associated with a preferred biocompatible matrix such as those described elsewhere herein. As will be appreciated by one of ordinary skill in the art, confluent, near confluent or post-confluent populations of cells are identifiable readily by a variety of techniques, the most common and widely-accepted of which is direct microscopic examination. Others include evaluation of cell number per surface area using standard cell counting techniques such as but not limited to a hemacytometer or coulter counter.

Additionally, for purposes of the present invention, endothelial-like cells include but are not limited to cells which emulate or mimic functionally and phenotypically confluent, near confluent or post-confluent endothelial cells as measured by the parameters set forth herein.

Thus, using the detailed description and guidance set forth below, the practitioner of ordinary skill in the art will appreciate how to make, use, test and identify operative embodiments of the implantable material disclosed herein. That is, the teachings provided herein disclose all that is necessary to make and use the present invention's implantable materials. And further, the teachings provided herein disclose all that is necessary to identify, make and use operatively equivalent cell-containing compositions. At bottom, all that is required is that equivalent cell-containing compositions are effective to treat, manage, modulate and/or ameliorate an affected site in accordance with the methods disclosed herein. As will be appreciated by the skilled practitioner, equivalent embodiments of the present composition can be identified using only routine experimentation together with the teachings provided herein.

In certain preferred embodiments, endothelial cells used in the implantable material of the present invention are isolated from the aorta of human cadaver donors. Each lot of cells is derived from a single donor or from multiple donors, tested extensively for endothelial cell purity, biological function, the presence of bacteria, fungi, known human pathogens and other adventitious agents. The cells are cryopreserved and banked using well-known techniques for later expansion in culture for subsequent formulation in biocompatible implantable materials.

Cell Preparation: As stated above, suitable cells can be obtained from a variety of tissue types and cell types. In certain preferred embodiments, human aortic endothelial cells used in the implantable material are isolated from the aorta of cadaver donors. In other embodiments, porcine aortic endothelial cells are isolated from normal porcine aorta by a similar procedure used to isolate human aortic endothelial cells. Each lot of cells can be derived from a single donor or from multiple donors, tested extensively for endothelial cell viability, purity, biological function, the presence of mycoplasma, bacteria, fungi, yeast, known human pathogens and other adventitious agents. The cells are further expanded, characterized and cryopreserved to form a working cell bank at the third to sixth passage using well-known techniques for later expansion in culture and for subsequent formulation in biocompatible implantable material.

The human or porcine aortic endothelial cells are prepared in T-75 flasks pre-treated by the addition of approximately 15 ml of endothelial cell growth media per flask. Human aortic endothelial cells are prepared in Endothelial Growth Media (EGM-2, Lonza, Basel, Switzerland). EGM-2 consists of Endothelial Cell Basal Media (EBM-2, Lonza) supplemented with EGM-2 singlequots, which contain 2% FBS. Porcine cells are prepared in EBM-2 supplemented with 5% FBS and 50 μg/ml gentamicin. The flasks are placed in an incubator maintained at approximately 37° C. and 5% CO₂/95% air, 90% humidity for a minimum of 30 minutes. One or two vials of the cells are removed from the −160° C. to −140° C. freezer and thawed at approximately 37° C. Each vial of thawed cells is seeded into two T-75 flasks at a density of approximately 3×10³ cells per cm², preferably, but no less than 1.0×10³ and no more than 7.0×10³; and the flasks containing the cells are returned to the incubator. After about 8-24 hours, the spent media is removed and replaced with fresh media. The media is changed every two to three days, thereafter, until the cells reach approximately 85-100% confluence preferably, but no less than 60% and no more than 100%. When the implantable material is intended for clinical application, only antibiotic-free media is used in the post-thaw culture of human aortic endothelial cells and manufacture of the implantable material of the present invention.

The endothelial cell growth media is then removed, and the monolayer of cells is rinsed with 10 ml of HEPES buffered saline (HEPES). The HEPES is removed, and 2 ml of trypsin is added to detach the cells from the surface of the T-75 flask. Once detachment has occurred, 3 ml of trypsin neutralizing solution (TNS) is added to stop the enzymatic reaction. An additional 5 ml of HEPES is added, and the cells are enumerated using a hemocytometer. The cell suspension is centrifuged and adjusted to a density of, in the case of human cells, approximately 2.0−1.75×10⁶ cells/ml using EGM-2 without antibiotics, or in the case of porcine cells, approximately 2.0−1.50×10⁶ cells/ml using EBM-2 supplemented with 5% FBS and 50 μg/ml gentamicin.

Biocompatible Matrix: According to the present invention, the implantable material comprises a biocompatible matrix. The matrix is permissive for cell growth and attachment to, on or within the matrix. The matrix is flexible and conformable. The matrix can be a solid, a semi-solid or flowable porous composition. For purposes of the present invention, flowable composition means a composition susceptible to administration using an injection or injection-type delivery device such as, but not limited to, a needle, a syringe or a catheter. Other delivery devices which employ extrusion, ejection or expulsion are also contemplated herein. Porous matrices are preferred. The matrix also can be in the form of a flexible planar form. The matrix also can be in the form of a gel, a foam, a suspension, a particle, a microcarrier, a microcapsule, or a fibrous structure. A preferred flowable composition is shape-retaining. A currently preferred matrix has a particulate form. The biocompatible matrix can comprise particles and/or microcarriers and the particles and/or microcarriers can further comprise gelatin, collagen, fibronectin, fibrin, laminin or an attachment peptide. One exemplary attachment peptide is a peptide of sequence arginine-glycine-aspartate (RGD).

The matrix, when implanted on an exterior or interior surface of an affected structure, can reside at the implantation site for at least about 7-90 days, preferably about at least 7-14 days, more preferably about at least 14-28 days, most preferably about at least 28-90 days before it bioerodes.

One preferred matrix is Gelfoam® (Pfizer, Inc., New York, N.Y.), an absorbable gelatin sponge (hereinafter “Gelfoam matrix”). Another preferred matrix is Surgifoam® (Johnson & Johnson, New Brunswick, N.J.), also an absorbable gelatin sponge. Gelfoam and Surgifoam matrices are porous and flexible surgical sponges prepared from a specially treated, purified porcine dermal gelatin solution.

According to another embodiment, the biocompatible matrix material can be a modified matrix material. Modifications to the matrix material can be selected to optimize and/or to control function of the cells, including the cells' phenotype (e.g., the inhibitory phenotype) as described above, when the cells are associated with the matrix. According to one embodiment, modifications to the matrix material include coating the matrix with attachment factors or adhesion peptides that enhance the ability of the cells to decrease extracellular matrix degradation, to decrease pathological angiogenesis, to decrease abnormal neovascularization, to increase TIMP production, to decrease inflammation, to increase heparan sulfate production, to increase prostacyclin production, and/or to increase TGF-β₁ and nitric oxide (NO) production.

According to another embodiment, the matrix is a matrix other than Gelfoam. Additional exemplary matrix materials include, for example, fibrin gel, alginate, polystyrene sodium sulfonate microcarriers, collagen coated dextran microcarriers, PLA/PGA and pHEMA/MMA copolymers (with polymer ratios ranging from 1-100% for each copolymer). According to one embodiment, a synthetic matrix material, for example, PLA/PGA, is treated with NaOH to increase the hydrophilicity of the material and, therefore, the ability of the cells to attach to the material. According to a preferred embodiment, these additional matrices are modified to include attachment factors or adhesion peptides, as recited and described above. Exemplary attachment factors include, for example, gelatin, collagen, fibronectin, fibrin gel, and covalently attached cell adhesion ligands (including for example RGD) utilizing standard aqueous carbodiimide chemistry. Additional cell adhesion ligands include peptides having cell adhesion recognition sequences, including but not limited to: RGDY, REDVY, GRGDF, GPDSGR, GRGDY and REDV.

Embodiments of Implantable Materials: As stated earlier, implantable material of the present invention can be a flexible planar form or a flowable composition. When in a flexible planar form, it can assume a variety of shapes and sizes, preferably a shape and size which conforms to a contoured interior or exterior surface of an affected structure or resected structure when situated at or adjacent to or in the vicinity of an affected or resected site. Examples of preferred configurations suitable for use in this manner are disclosed in co-owned international patent application PCT/US05/43967 filed on Dec. 6, 2005 (also known as Attorney Docket No. ELV-002PC), the entire contents of which are herein incorporated by reference.

Flowable Composition: In certain embodiments contemplated herein, the implantable material of the present invention is a flowable composition comprising a particulate biocompatible matrix which can be in the form of a gel, a foam, a suspension, a particle, a microcarrier, a microcapsule, macroporous beads, or other flowable material. The current invention contemplates any flowable composition that can be administered with an injection-type delivery device. For example, a delivery device that can navigate the interior of an affected or resected structure, or a percutaneous injection-type delivery device, is suitable for this purpose as described below. The flowable composition is preferably a shape-retaining composition. Thus, an implantable material comprising cells in, on or within a flowable-type particulate matrix as contemplated herein can be formulated for use with any injectable delivery device ranging in internal diameter from about 18 gauge to about 26 gauge and capable of delivering about 50 mg of flowable composition comprising particulate material containing preferably about 1 million cells in about 1 to about 3 ml of flowable composition.

According to a currently preferred embodiment, the flowable composition comprises a biocompatible particulate matrix such as Gelfoam® particles, Gelfoam® powder, or pulverized Gelfoam® (Pfizer Inc., New York, N.Y.) (hereinafter “Gelfoam particles”), a product derived from porcine dermal gelatin. According to another embodiment, the particulate matrix is Surgifoam™ (Johnson & Johnson, New Brunswick, N.J.) particles, comprised of absorbable gelatin powder. According to another embodiment, the particulate matrix is Cytodex-3 (Amersham Biosciences, Piscataway, N.J.) microcarriers, comprised of denatured collagen coupled to a matrix of cross-linked dextran. According to a further embodiment, the particulate matrix is CultiSpher-G (Percell Biolytica AB, Astorp, Sweden) microcarrier, comprised of porcine gelatin. According to another embodiment, the particulate matrix is a macroporous material. According to one embodiment, the macroporous particulate matrix is CytoPore (Amersham Biosciences, Piscataway, N.J.) microcarrier, comprised of cross-linked cellulose which is substituted with positively charged N,N,-diethylaminoethyl groups.

According to alternative embodiments, the biocompatible implantable particulate matrix is a modified biocompatible matrix. Modifications include those described above for an implantable matrix material.

Related flowable compositions suitable for use to manage the development and/or progression of healing in affected sites in accordance with the present invention are disclosed in co-owned international patent application PCT/US05/43844 filed on Dec. 6, 2005 (also known as Attorney Docket No. ELV-009PC), the entire contents of which are herein incorporated by reference.

Preparation of Implantable Material: Prior to Cell Seeding, the biocompatible matrix is re-hydrated by the addition of EGM-2 without antibiotics at approximately 37° C. and 5% CO₂/95% air for 12 to 24 hours. The implantable material is then removed from their re-hydration containers and placed in individual tissue culture dishes. The biocompatible matrix is seeded at a preferred density of approximately 1.5−2.0×10⁵ cells (1.25−1.66×10⁵ cells/cm³ of matrix) and placed in an incubator maintained at approximately 37° C. and 5% CO₂/95% air, 90% humidity for 3-4 hours to facilitate cell attachment. The seeded matrix is then placed into individual containers (Evergreen, Los Angeles, Calif.), each fitted with a cap containing a 0.2 μm filter with EGM-2 and incubated at approximately 37° C. and 5% CO₂/95% air. Alternatively, three pieces of the seeded matrix can be placed into a 150 mL bottle. The media is changed every two to three days, thereafter, until the cells have reached confluence. The cells in one preferred embodiment are preferably passage 6, but cells of fewer or more passages can be used.

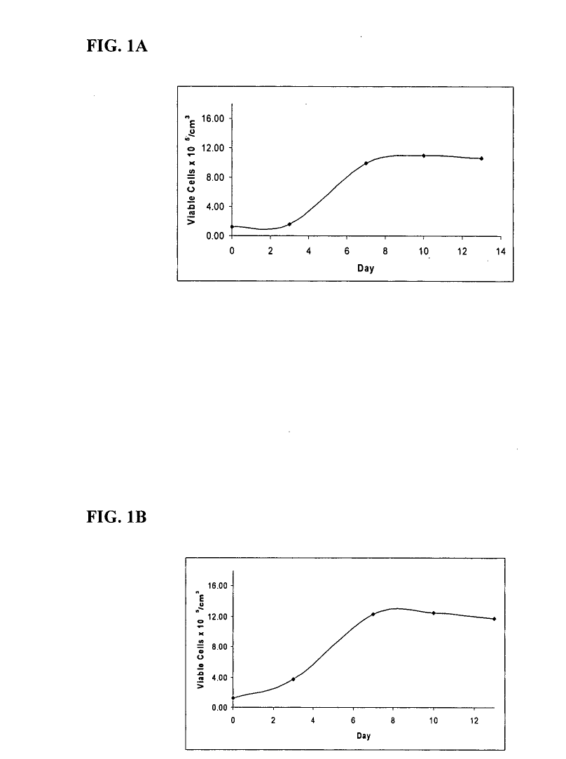

Cell Growth Curve and Confluence: A sample of implantable material is removed on or around days 3 or 4, 6 or 7, 9 or 10, and 12 or 13, the cells are counted and assessed for viability, and a growth curve is constructed and evaluated in order to assess the growth characteristics and to determine whether confluence, near confluence or post-confluence has been achieved. Representative growth curves from two preparations of implantable material comprising porcine aortic endothelial cell implanted lots are presented in FIGS. 1A and 1B. In these examples, the implantable material is in a flexible planar form. Generally, one of ordinary skill will appreciate the indicia of acceptable cell growth at early, mid- and late time points, such as observation of an increase in cell number at the early time points (when referring to FIG. 1A, between about days 2-6), followed by a near confluent phase (when referring to FIG. 1A, between about days 6-8), followed by a plateau in cell number once the cells have reached confluence as indicated by a relatively constant cell number (when referring to FIG. 1A, between about days 8-10) and maintenance of the cell number when the cells are post-confluent (when referring to FIG. 1A, between about days 10-14). For purposes of the present invention, cell populations which are in a plateau for at least 72 hours are preferred.

Cell counts are achieved by complete digestion of the aliquot of implantable material with a solution of 0.5 mg/ml collagenase in a CaCl₂ solution. After measuring the volume of the digested implantable material, a known volume of the cell suspension is diluted with 0.4% trypan blue (4:1 cells to trypan blue) and viability assessed by trypan blue exclusion. Viable, non-viable and total cells are enumerated using a hemacytometer. Growth curves are constructed by plotting the number of viable cells versus the number of days in culture. Cells are shipped and implanted after reaching confluence.

For purposes of the present invention, confluence is defined as the presence of at least about 4×10⁵ cells/cm³ when in a flexible planar form of the implantable material (1.0×4.0×0.3 cm), and preferably about 7×10⁵ to 1×10⁶ total cells per aliquot (50-70 mg) when in a flowable composition. For both, cell viability is at least about 90% preferably but no less than 80%. If the cells are not confluent by day 12 or 13, the media is changed, and incubation is continued for an additional day. This process is continued until confluence is achieved or until 14 days post-seeding. On day 14, if the cells are not confluent, the lot is discarded. If the cells are determined to be confluent after performing in-process checks, a final media change is performed. This final media change is performed using EGM-2 without phenol red and without antibiotics. Immediately following the media change, the tubes are fitted with sterile plug seal caps for shipping.

Evaluation of Functionality and Phenotype: For purposes of the invention described herein, the implantable material is further tested for indicia of functionality and phenotype prior to implantation. For example, conditioned media are collected during the culture period to ascertain levels of heparan sulfate, transforming growth factor-β₁ (TGF-β₁), basic fibroblast growth factor (b-FGF), tissue inhibitors of matrix metalloproteinases (TIMP), and nitric oxide (NO) which are produced by the cultured endothelial cells. In certain preferred embodiments, the implantable material can be used for the purposes described herein when total cell number is at least about 2, preferably at least about 4×10⁵ cells/cm³ of implantable material; percentage of viable cells is at least about 80-90%, preferably ≧90%, most preferably at least about 90%; heparan sulfate in conditioned media is at least about 0.23-1.0, preferably at least about 0.5 microg/mL/day; TGF-β₁ in conditioned media is at least about 200-300 picog/mL/day, preferably at least about 300 picog/ml/day; b-FGF in conditioned media is below about 200 picog/ml, preferably no more than about 400 picog/ml; TIMP-2 in conditioned media is at least about 5.0-10.0 ng/mL/day, preferably at least about 8.0 ng/mL/day; NO in conditioned media is at least about 0.5-3.0 μmol/L/day, preferably at least about 2.0 μmol/L/day.

Heparan sulfate levels can be quantified using a routine dimethylmethylene blue-chondroitinase ABC digestion spectrophotometric assay. Total sulfated glycosaminoglycan (GAG) levels are determined using a dimethylmethylene blue (DMB) dye binding assay in which unknown samples are compared to a standard curve generated using known quantities of purified chondroitin sulfate diluted in collection media. Additional samples of conditioned media are mixed with chondroitinase ABC to digest chondroitin and dermatan sulfates prior to the addition of the DMB color reagent. All absorbances are determined at the maximum wavelength absorbance of the DMB dye mixed with the GAG standard, generally around 515-525 nm. The concentration of heparan sulfate per day is calculated by multiplying the percentange heparan sulfate calculated by enzymatic digestion by the total sulfated glycosaminoglycan concentration in conditioned media samples. Chondroitinase ABC activity is confirmed by digesting a sample of purified 100% chondroitin sulfate and a 50/50 mixture of purified heparan sulfate and chondroitin sulfate. Conditioned medium samples are corrected appropriately if less than 100% of the purified chondroitin sulfate is digested. Heparan sulfate levels may also be quantified using an ELISA assay employing monoclonal antibodies.

TGF-β₁, TIMP, and b-FGF levels can be quantified using an ELISA assay employing monoclonal or polyclonal antibodies, preferably polyclonal. Control collection media can also be quantified using an ELISA assay and the samples corrected appropriately for TGF-β₁, TIMP, and b-FGF levels present in control media.

Nitric oxide (NO) levels can be quantified using a standard Griess Reaction assay. The transient and volatile nature of nitric oxide makes it unsuitable for most detection methods. However, two stable breakdown products of nitric oxide, nitrate (NO₃) and nitrite (NO₂), can be detected using routine photometric methods. The Griess Reaction assay enzymatically converts nitrate to nitrite in the presence of nitrate reductase. Nitrite is detected colorimetrically as a colored azo dye product, absorbing visible light in the range of about 540 nm. The level of nitric oxide present in the system is determined by converting all nitrate into nitrite, determining the total concentration of nitrite in the unknown samples, and then comparing the resulting concentration of nitrite to a standard curve generated using known quantities of nitrate converted to nitrite.

The earlier-described preferred inhibitory phenotype is assessed using the quantitative heparan sulfate, TGF-β₁, TIMP, NO and/or b-FGF assays described above, as well as quantitative in vitro assays of smooth muscle cell growth, and inhibition of thrombosis as follows. For purposes of the present invention, implantable material is ready for implantation when one or more of these alternative in vitro assays confirm that the implantable material is exhibiting the preferred inhibitory phenotype.

To evaluate inhibition of smooth muscle cell growth in vitro, the magnitude of inhibition associated with cultured endothelial cells is determined. Porcine or human aortic smooth muscle cells are sparsely seeded in 24 well tissue culture plates in smooth muscle cell growth medium (SmGM-2, Lonza). The cells are allowed to attach for 24 hours. The media is then replaced with smooth muscle cell basal media (SmBM) containing 0.2% FBS for 48-72 hours to growth arrest the cells. Conditioned media is prepared from post-confluent endothelial cell cultures, diluted 1:1 with 2×SMC growth media and added to the cultures. A positive control for inhibition of smooth muscle cell growth is included in each assay. After three to four days, the number of cells in each sample is enumerated using a Coulter Counter or using a colorimetric assay after the addition of a dye. The effect of conditioned media on smooth muscle cell proliferation is determined by comparing the number of smooth muscle cells per well immediately before the addition of conditioned media with that after three to four days of exposure to conditioned media, and to control media (standard growth media with and without the addition of growth factors). The magnitude of inhibition associated with the conditioned media samples are compared to the magnitude of inhibition associated with the positive control. According to a preferred embodiment, the implantable material is considered inhibitory if the conditioned media inhibits about 20% of what the heparin control is able to inhibit.

To evaluate inhibition of thrombosis in vitro, the level of heparan sulfate associated with the cultured endothelial cells is determined. Heparan sulfate has both anti-proliferative and anti-thrombotic properties. Using either the routine dimethylmethylene blue-chondroitinase ABC digestion spectrophotometric assay or an ELISA assay, both assays are described in detail above, the concentration of heparan sulfate is calculated. The implantable material can be used for the purposes described herein when the heparan sulfate in the conditioned media is at least about 0.23-1.0, preferably at least about 0.5 microg/mL/day.

Another method to evaluate inhibition of thrombosis involves determining the magnitude of inhibition of platelet aggregation in vitro associated with platelet rich-plasma or platelet concentrate (Research Blood Components, Brighton, Mass.). Conditioned media is prepared from post-confluent endothelial cell cultures and added to aliquots of the platelet concentrate. A platelet aggregating agent (agonist) is added to the platelets seeded into 96 well plates as control. Platelet agonists commonly include arachidonate, ADP, collagen type I, epinephrine, thrombin (Sigma-Aldrich Co., St. Louis, Mo.) or ristocetin (available from Sigma-Aldrich Co., St. Louis, Mo.). An additional well of platelets has no platelet agonist or conditioned media added, to assess for baseline spontaneous platelet aggregation. A positive control for inhibition of platelet aggregation is also included in each assay. Exemplary positive controls include aspirin, heparin, indomethacin (Sigma-Aldrich Co., St. Louis, Mo.), abciximab (ReoPro®, Eli Lilly, Indianapolis, Ind.), tirofiban (Aggrastat®, Merck & Co., Inc., Whitehouse Station, N.J.) or eptifibatide (Integrilin®, Millennium Pharmaceuticals, Inc., Cambridge, Mass.). The resulting platelet aggregation of all test conditions are then measured using a plate reader and the absorbance read at 405 nm. The platelet reader measures platelet aggregation by monitoring optical density. As platelets aggregate, more light can pass through the specimen. The platelet reader reports results in absorbance, a function of the rate at which platelets aggregate. Aggregation is assessed as maximal aggregation between 6-12 minutes after the addition of the agonist. The effect of conditioned media on platelet aggregation is determined by comparing maximal agonist aggregation before the addition of conditioned medium with that after exposure of platelet concentrate to conditioned medium, and to the positive control. Results are expressed as a percentage of the baseline. The magnitude of inhibition associated with the conditioned media samples are compared to the magnitude of inhibition associated with the positive control. According to a preferred embodiment, the implantable material is considered regulatory if the conditioned media inhibits thrombosis by at least about 20% of the control, more preferably by at least about 40% of the control, and most preferably by at least about 60% of the control.

To evaluate regulation of angiogenesis in vitro, the vascular tube formation Matrigel plug assay of Javaherian et al. was used to evaluate the tube formation density of Matrigel sections. Javaherian et al. J. Bio. Chem. 277:45211-45218 (2002). Multiwell dishes (24 well) were coated with 250 μl Matrigel, an ECM preparation from the Engelbreth-Holm-Swarm tumor (BD PharMingen), at 4° C. and incubated at 37° C. for 30-60 minutes for the plugs to form. Human umbilical vein endothelial cells (HUVEC, Lonza BioSciences, Basel, Switzerland) were seeded at 50,000-100,000 cells/mL in 0.5 mL EGM-2-MV medium (Lonza BioSciences). Conditioned media collected from samples of implantable material or the implantable material in a co-culture system as an implantable material insert was applied to the Matrigel either at the time of plating (t=0) or after various HUVEC incubation times at 37° C. (t=2, 4, 8, 12 or 16 hours).

The density of endothelial cell tubes formed within the Matrigel was quantified by manual counting in triplicate of low power fields (40×). Samples of the biocompatible matrix without cells (or nothing applied to the Matrigel) were used as the negative control. Any know anti-angiogenic drug can be used as the positive control (for example, thrombospondin-1, endostatin or avastatin).

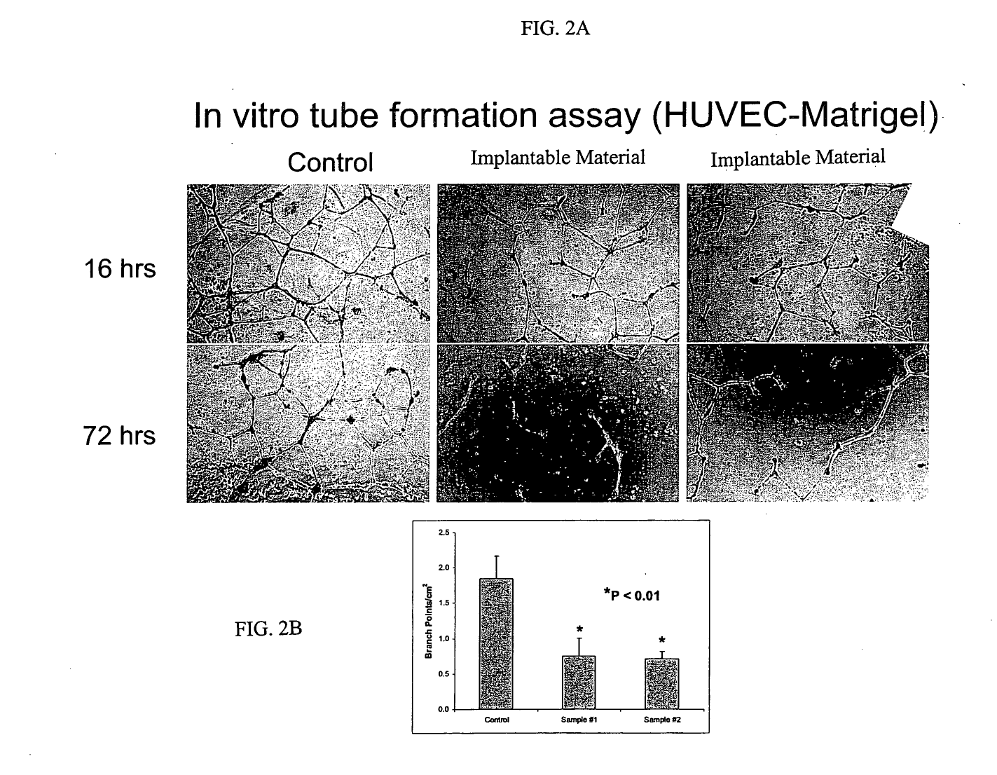

FIG. 2A depicts photographs of HUVEC tube formation in Matrigel with and without treatment with the implantable material of the present invention. FIG. 2B is a graphical representation of the tube formation density following administration of conditioned media or the implantable material. With reference to FIGS. 2A and 2B, according to this method, the implantable material resulted in a reduction in the density of tube formation in the Matrigel compared to the control. According to this embodiment, the Matrigel-HUVECs were allowed to incubate with conditioned medium from the implantable material for 72 hours. At 16 and 72 hours following administration of the conditioned media, significant tube formation remains within the untreated Matrigel sample. In the implantable material samples, on the other hand, the density of tube formation is significantly reduced. Accordingly, the implantable material is able to inhibit angiogenesis in the Matrigel relative to the control.

When ready for implantation, the planar form of implantable material is supplied in final product containers, each preferably containing a 1×4×0.3 cm (1.2 cm³), sterile implantable material with preferably approximately 5−8×10⁵ or preferably at least about 4×10⁵ cells/cm³, and at least about 90% viable cells (for example, human aortic endothelial cells derived from a single cadaver donor) per cubic centimeter implantable material in approximately 45-60 ml, preferably about 50 ml, endothelial growth medium (for example, endothelial growth medium (EGM-2), containing no phenol red and no antibiotics). When porcine aortic endothelial cells are used, the growth medium is also EBM-2 containing no phenol red, but supplemented with 5% FBS and 50 μg/ml gentamicin.

In other preferred embodiments, the flowable composition (for example, a particulate form biocompatible matrix) is supplied in final product containers, including, for example, sealed tissue culture containers modified with filter caps or pre-loaded syringes, each preferably containing about 50-60 mg of flowable composition comprising about 7×10⁵ to about 1×10⁶ total endothelial cells in about 45-60 ml, preferably about 50 ml, growth medium per aliquot.

Shelf-Life of Implantable Material: The implantable material of the present invention comprising a confluent, near-confluent or post-confluent population of cells can be maintained at room temperature in a stable and viable condition for at least two weeks. Preferably, such implantable material is maintained in about 45-60 ml, more preferably about 50 ml per implantable material, of transport media with or without additional FBS or VEGF. Transport media comprises EGM-2 media without phenol red. FBS can be added to the volume of transport media up to about 10% FBS, or a total concentration of about 12% FBS. However, because FBS must be removed from the implantable material prior to implantation, it is preferred to limit the amount of FBS used in the transport media to reduce the length of rinse required prior to implantation. VEGF can be added to the volume of transport media up to a concentration of about 3-4 ng/mL.

Cryopreservation of Implantable Material: The implantable material of the present invention can be cryopreserved for storage and/or transport to the implantation site without diminishing its clinical potency or integrity upon eventual thaw. Preferably, implantable material is cryopreserved in a 15 ml cryovial (Nalgene®, Nalge Nunc Intl, Rochester, N.Y.) in a solution of about 5 ml CryoStor CS-10 solution (BioLife Solutions, Oswego, N.Y.) containing about 10% DMSO, about 2-8% Dextran and about 20-75% FBS and/or human serum. Cryovials are placed in a cold iso-propanol water bath, transferred to an −80° C. freezer for 4 hours, and subsequently transferred to liquid nitrogen (−150° C. to −165° C.).

Cryopreserved aliquots of the implantable material are then slowly thawed at room temperature for about 15 minutes, followed by an additional approximately 15 minutes in a room temperature water bath. The material is then washed about 3 times in about 200-250 mL saline, lactated ringers or EBM. The three rinse procedures are conducted for about 5 minutes at room temperature. The material is then implanted.

To determine the bioactivity of the thawed material, following the thaw and rinse procedures, the cryopreserved material is allowed to rest for about 48 hours in about 10 ml of recovery solution. For porcine endothelial cells, the recovery solution is EBM-2 supplemented with 5% FBS and 50 mg/ml gentamicin at 37° C. in 5% CO₂; for human endothelial cells, the recovery solution is EGM-2 with or without antibiotics. Further post-thaw conditioning can be carried out for at least another 24 hours prior to use and/or packaging for storage or transport.

Immediately prior to implantation, the transport or cryopreservation medium is decanted and the implantable material is rinsed in about 250-500 ml sterile saline (USP). The medium in the final product contains a small amount of FBS to maintain cell viability during transport to a clinical site if necessary. The FBS has been tested extensively for the presence of bacteria, fungi and other viral agents according to Title 9 CFR: Animal and Animal Products. A rinsing procedure is employed just prior to implantation, which decreases the amount of FBS transferred preferably to between 0-60 ng per implant, but preferably no more than 1-2 μg per implant.

The total cell load per human patient will be preferably approximately 1.6−2.6×10⁴ cells per kg body weight, but no less than about 2×10³ and no more than about 2×10⁶ cells per kg body weight.

Administration of Implantable Material: The implantable material of the present invention when in a flowable composition comprises a particulate biocompatible matrix and cells, preferably endothelial cells, more preferably vascular endothelial cells, which are about 90% viable at a preferred density of about 0.8×10⁴ cells/mg, more preferred of about 1.5×10⁴ cells/mg, most preferred of about 2×10⁴ cells/mg, and which can produce conditioned media containing heparan sulfate at least about 0.23-1.0, preferably at least about 0.5 microg/mL/day, TGF-β₁ at least about 200-300 picog/ml/day, preferably at least about 300 picog/ml/day, and b-FGF below about 200 picog/ml and preferably no more than about 400 picog/ml; TIMP-2 in conditioned media is at least about 5.0-10.0 ng/mL/day, preferably at least about 8.0 ng/mL/day; NO in conditioned media is at least about 0.5-3.0 μmol/L/day, preferably at least about 2.0 μmol/L/day; and, display the earlier-described inhibitory phenotype.

For purposes of the present invention generally, administration of the implantable particulate material is localized to a site in the vicinity of, adjacent to or at an affected site. The site of deposition of the implantable material is an interior or exterior surface of an affected structure or at an adjacent or surrounding tissue. As contemplated herein, localized deposition can be accomplished as follows.

In a particularly preferred embodiment, the flowable composition is first administered percutaneously, entering the patient's body near the affected structure and then deposited on an interior surface of an affected joint, resection or other cavity, exterior surface of an affected structure, directly in contact with the stroma or an interstitial site adjacent to or surrounding the affected or treated site using a suitable needle, catheter or other suitable percutaneous delivery device. Alternatively, the flowable composition is delivered percutaneously using a needle, catheter or other suitable delivery device in conjunction with an identifying step to facilitate delivery to a desired site. The identifying step can occur prior to or coincident with percutaneous delivery. The identifying step can be accomplished using physical examination, ultrasound, and/or CT scan, to name but a few. The identifying step is optionally performed and not required to practice the methods of the present invention.

The flowable composition can also be administered intraluminally, through a tubular structure adjacent to or connected to an affected structure. For example, the composition can be delivered by any device able to be inserted within a tubular structure. In this instance, such an intraluminal delivery device is equipped with a traversing or penetrating device which traverses or penetrates the luminal wall of the tubular structure to reach the interior or exterior surface of an affected structure. The flowable composition is then deposited on a surface of the affected structure or adjacent or surrounding tissue.

The traversing or penetrating devices contemplated herein can permit, for example, a single point of delivery or a plurality of delivery points arranged in a desired geometric configuration to accomplish delivery of flowable composition to a surface of an affected structure without disrupting the site. A plurality of delivery points can be arranged, for example, in a single circle, in concentric circles, or a linear array arrangement to name but a few.

According to a preferred embodiment of the invention, the penetrating device is inserted via the interior surface of a structure either proximal or distal to the site of treatment. In some clinical subjects, insertion of the penetrating device at or near the site of the treatment could disrupt or lead to further damage of the affected structure. Accordingly, in such subjects, care should be taken to insert the penetrating device at a location a distance from the affected structure, preferably a distance determined by the clinician governed by the specific circumstances at hand.

Preferably, flowable composition is deposited on an interior or exterior surface of an affected structure, either at the site to be treated, or adjacent to or in the vicinity of the site. The composition can be deposited in a variety of locations relative to an affected site, for example, at the site, contacting the stroma, surrounding the site or adjacent to the site. According to a preferred embodiment, an adjacent site is within about 0 cm to 2 cm of the affected site. In another preferred embodiment, a site is within about 2 cm to 4 cm; in yet another preferred embodiment, a site is within about 4 cm to 6 cm. In another preferred embodiment, a site is within about 6 cm to 10 cm. Alternatively, an adjacent site is any other clinician-determined adjacent location where the deposited composition is capable of exhibiting a desired effect on an affected site in the proximity of the site to be treated.

In another embodiment, the flowable composition is delivered directly to a surgically-exposed interior or exterior surface at, adjacent to or in the vicinity of an affected structure. In this case delivery is guided and directed by direct observation of the site. Also in this case, delivery can be aided by coincident use of an identifying step as described above. Again, the identifying step is optional.

According to another embodiment of the invention, the flexible planar form of the implantable material is delivered locally to a surgically-exposed exterior or interior surface or cavity at, adjacent to or in the vicinity of an affected site. In one case, at least one piece of the implantable material is applied to the interior of a cavity; it need only conform to and contact a surface of the site and be implanted in an amount effective to treat the site.

According to one embodiment, alone or optionally following surgical tumor resection, the resection site is administered radiation therapy to irradiate the tumor and/or remaining cancer cells at or in the vicinity of the resection site.

According to another embodiment, alone or optionally following surgical tumor resection, the patient is administered chemotherapy to kill the tumor and/or remaining cancer cells at or in the vicinity of the resection site. According to either of these embodiments, following completion of the radiation therapy and/or chemotherapy, the implantable material is delivered to the resection cavity.

According to this method, the implantable material is used to treat, ameliorate, manage and/or reduce the effects of tumor resection, radiation therapy and/or chemotherapy on the resection cavity and/or surrounding tissues.

### EXAMPLES

### 1. Vascular Injury in Pigs

This study exemplifies use of the present invention's materials and methods to modulate pathological angiogenesis and abnormal neovascularization. The experimental model chosen for such exemplification is neovascularization following vascular injury and trauma induced by surgical intervention. Furthermore, this example provides experimental protocols for testing and using a preferred embodiment of the present invention to reduce or modulate indicia of abnormal neovascularization including neovascularization, vascular density, and expression levels of MMPs following an intervention to a vascular tubular structure, for example, introduction of an AV graft, in animal test subjects.

Using standard surgical procedures, an AV graft was created between the carotid artery and the jugular vein. Implantable material was then disposed in the perivascular space adjacent to each surgically created AV graft anastomosis; the details of one exemplary procedure are set forth below. As described earlier, the placement and configuration of implantable material can be varied. In this study, the implantable material was in a flexible planar form.

Specifically, the study included 20 porcine test subjects undergoing AV graft surgery. Conventional AV graft surgery procedures were performed according to standard operative techniques. Implantable material was applied to the AV graft anastomoses and surrounds as described below after the graft surgery was completed and flow through the graft was established.

For each test subject undergoing AV graft surgery, one six-millimeter internal diameter PTFE graft was placed between the left common carotid artery and right external jugular vein of the test subject. An oblique end-to-side anastomosis was created at each end of the graft using a running 6-0 prolene suture. All test subjects received intra-operative heparin and administered daily aspirin following surgery.

Ten of the test subjects received implantable material comprising aortic endothelial cells on the day of surgery. Five such implants were applied to each test subject. Two implants were wrapped around each of the two anastomotic sites. In this circumstance, one end of the first piece of implantable material was passed under the anastomotic segment until the middle of the implant was at the point where the vessel and graft meet. The second piece of implantable material was then wrapped in a direction opposite that of the first piece, placed on top of the anastomotic segment and the ends tucked under the anastomosis. Both ends were then wrapped around the suture line keeping the implant centered over the suture line. The ends overlapped minimally to secure the material in place. An additional single implant was placed longitudinally along the length of the proximal venous segment starting at the anastomosis, of each test subject. The implant did not completely wrap around the circumference of the vein.

Ten test subjects received control implants without cells, wrapped around the anastomotic sites and placed on the proximal venous segment of the graft on the day of surgery. The total cell load based on body weight was approximately 2.5×10⁵ cells per kg.

Surgical Procedure. Bilateral 8-cm neck incisions were made over the sternocleidomastoid muscle on each side of the neck. Using these incisions, the left common carotid artery was isolated followed by the right external jugular vein. Approximately 4-8 cm segments of vein and artery were freed from surrounding tissues and all tributaries off the vein were ligated with 3-0 silk sutures. A 6-mm internal diameter PTFE graft (Atrium Medical Corp., Hudson, N.H.) was tunneled in a subcutaneous tract between the two incisions. The isolated jugular vein was clamped and a 10-mm venotomy was made. The vein was irrigated with heparinized saline solution and an oblique end-to-side anastomosis was made between the vein graft using a running 6-0 prolene suture. The average graft length was 18.6±0.9 cm. Once fashioned, the venous clamp was removed, the graft flushed with heparin-saline solution and re-clamped. The left carotid artery was then clamped and an 8-mm arteriotomy performed. The artery was flushed with heparinized saline solution and an oblique end-to-side anastomosis was made between the artery and graft using 6-0 proline suture. Vascular clamps were removed and flow through the graft was confirmed by the physical palpation of thrill in the graft. Hemostatis of each vascular anastomosis was confirmed and on rare occasion an additional 6-0 prolene suture was placed in an interrupted fashion at the point of anastomotic bleeding.

Following completion of the anastomoses, the PTFE arteriovenous graft was positioned to prevent kinking. The PTFE arteriovenous graft was percutaneously cannulated with a 23-gauge butterfly needle just distal to the carotid artery-graft anastomosis. To confirm placement, blood was aspirated into the system with a 10 cc syringe. The system was then flushed with 10 cc's of saline. A C-arm fluoroscope was then placed over the neck of the study animal so that the venous-graft anastomosis and the venous outflow tract could be visualized. Under continuous fluoroscopy, 10-15 cc's of iodinated contrast (Renograffin, full strength) was injected.

After completion of the angiography, the anastomotic sites were wrapped in a wet 4″×4″ gauze sponge. Pressure was maintained on the anastomotic sites for a period of approximately 5 minutes, before removing the gauze sponges and inspecting the anastomotic sites. If hemostasis had not yet been achieved, as was evidenced by oozing of blood, the site was again wrapped for another 5 minutes. Additional sutures were placed at the discretion of the surgeon if the hemorrhage from the site was severe. Once hemostasis had been achieved, the neck wound was filled with sterile saline and flow probe analysis performed at the distal venous outflow tract using a 6-mm Transonic flow probe. The saline was removed, if necessary, and the anastomoses made as dry as possible and treated with either implantable material comprising aortic endothelial cells or control implants. Sites were not treated with either type of implant until all bleeding had been controlled, flow through the graft confirmed and the area made as dry as possible. When complete, the wound was closed in layers and the animal was allowed to recover from anesthesia.

Heparin was administered prior to surgery as a 100 U/kg bolus injection plus a 35 U/kg/hr continuous infusion and maintained until the end of surgery. Additional bolus doses (100U/kg) were administered, as necessary to maintain ACTs 200 seconds.

Graft Patency. AV graft patency was confirmed by access flow measurements using color-flow Doppler ultrasound and Transonic flow probe (Transonic Systems, Inc., Ithaca, N.Y.) immediately after surgery, 3-7 days post surgery and once per week thereafter. Grafts were monitored closely for blood flow.

Pathology Procedures. Animal test subjects were anesthetized using sodium pentobarbital (65 mg/kg, IV). Graft patency was determined prior to necropsy by cine angiography as described above. After completion of the angiography, the grafts/anastomoses were perfused with PBS followed by formalin.

Histology. Half of the animal test subjects (5 cell engrafted implant subjects; 5 control implant subjects) were euthanized 3 days following surgery. The remaining animal test subjects (5 cell engrafted implant subjects; 5 control implant subjects) were euthanized one month following surgery.

A limited necropsy, defined as the macroscopic examination of the administration site, including all anastomotic and proximal venous sites, and surrounding tissue including draining lymph nodes was performed on all test subjects. Tissue from major organs, including brain, lungs, kidneys, liver, heart and spleen, were collected and saved for all test subjects euthanized at one month following surgery. The organs were to be analyzed only if unusual findings arose from macroscopic examination of the external surface of the body or from the microscopic examination of administration sites and surrounding tissue. No unusual findings arose that warranted further examination of the major organs in any of the animals enrolled into the study.

All AV graft anastomotic sites and surrounding tissues, including 5-cm segments each of the anastomosed vein and artery, were trimmed, fixed in 10% formalin (or equivalent) and embedded in glycolmethacrylate (or equivalent). Using approximately 3μm-thick sections cut with a C-profile stainless steel knife (or equivalent), sections were prepared from at least three regions: the vein graft anastomosis, the graft-artery anastomosis, and the venous outflow tract. Three sections were made transversely through the vein graft anastomosis. Five sections were made through the venous outflow tract (therefore covering 1.5-cm of outflow vein). Three sections were made through the graft-artery anastomosis at 1-mm intervals. These sections were mounted on gelatin-coated (or equivalent) glass slides and stained with hematoxylin and eosin or Verhoeff's elastin stain.

Each section was evaluated for the presence and/or extent of neovascularization. Scores were assigned for each variable on a scale of 0 through 4 (0=no significant changes; 1=minimal; 2=mild; 3=moderate; and 4=severe). Representative grading criteria for neovascularization findings are presented below in Table 1.

TABLE 1
Grading Criteria for Neovascularization Findings

| | 0 | 1 | 2 | 3 | 4 |
|---|---|---|---|---|---|
| Neovascularization | 0 | Focal ≦ 5 | 6-15 vessels | 16-25 | >25 |
| (per 20x HPF) | | vessels | | vessels | vessels |
| | | | | | and/or |
| | | | | | Diffuse |

HPF = High Powered Field

To examine the effects of the perivascular implants on matrix metalloproteinase (MMP) expression, venous tissue sections were subjected to immunohistochemical analysis. Five micrometer paraffin sections were cut and antigen retrieval performed by heating the sections for 20 minutes in high pH Target Retrieval Solution (Dako USA, Carpinteria, Calif.). The slides were covered with Peroxidase Block (Dako USA) for 5 minutes to quench endogenous peroxidase activity. Primary murine anti-human MMP-2 (1:250 dilution, Chemicon International, Inc. Ternecula, Calif.) was applied for 45 minutes at room temperature and Primary rabbit anti-human MMP-9 (1:250 dilution, Chemicon International, Inc. Ternecula, Calif.) was applied for 60 minutes at room temperature. All slides were counterstained with Mayer's hematoxylin (Sigma Chemical Co.). Porcine liver was used as a positive control and mouse IgG1 or rabbit IgG were used as negative control. For every specimen, at least 6 non-overlapping fields were analyzed per section. For quantitative assessment of positive MMP staining, randomly selected areas were imaged using an Olympus BX60 microscope. Digital images (200× magnification) were captured and analyzed using Image-Pro Plus 6.0 software (Media Cybernetics, Silver Spring, Md.). Each area of interest (e.g. intima, media and adventitia) was highlighted and positive staining was quantified by color segmentation. The results were expressed as percentage of positive stained area (positive area in mm² over total area in mm²).

Results for Animal Subjects. Placement of the implantable material of the present invention at a site at or adjacent to a tubular or non-tubular tissue structure is effective to diminish neovascularization which follows a disruption of the tissue, for example, following a surgical intervention. Administration of the implantable material of the present invention at a site at or adjacent to a surgically treated tubular or non-tubular tissue structure decreases abnormal neovascularization in the treated tissue structures. Furthermore, the implantable material reduces MMP expression and/or activation of the tubular or non-tubular tissue structures.

Evidence of neovascularization was observed in both groups at both time points. Adventitial neovascularization is characterized by the ingrowth into tissue of newly forming small vessels (capillaries) which at its greatest extent has an orderly pattern consistent with granulation tissue (vessels perpendicular to fibroblasts), either reorganization of injured tissue or new growth into materials that would not ordinarily contain vessels. Both acute and chronic neovascularization was decreased in veins treated with the implantable material when compared to veins administered the control material (an average difference of 1 severity point in both cases, Table 2).

TABLE 2
Summary of Average Severity of
Neovascularization - Adventitial Venous Sites

Neovascularization

| Time (Days) | Control | Implantable Material |
|---|---|---|
| 3 (Acute) | 1.2 ± 0.2 | 0.6 ± 0.08^{§} |
| 28 (Chronic) | 3.0 ± 0.3 | 2.1 ± 0.2^{§} |

^{§}P < 0.05 compared to controls
Average severity = (severity of group/incidence)
0 = none,
1 = minimal;
2 = mild;
3 = moderate;
4 = severe

The implantable material of the present invention also reduced expression of matrix metalloproteinases in animals treated with the implantable material of the present invention. Immunohistochemical analysis of MMP-2 and MMP-9 positive cells in the total vessel, intima, media and adventitia 3-days and 1-month after surgery revealed reduced expression of MMPs in veins treated with the implantable material compared to veins administered the control material.

In the control group, significant MMP-2 positive cells were observed in the adventitia, media and intima at day 3. MMP-2 positive cells were observed in tissue sections of animals administered the control material at a level of 11.2±1.0% in the adventitia; 4.4±0.6% in the media; and 2.1±0.2% in the intima. In animals receiving the control material, positive staining for MMP-2 was predominantly located in the adventitia. At 1-month, the amount of staining in animals administered the control material decreased in the adventitia (5.7±0.9%) but remained increased in the media (4.9±1.0%) and the intima (2.6±0.8%).

In the group treated with the implantable material, decreased expression of MMP-2 was observed in the adventitia, media and intima of vessels at day 3. MMP-2 positive cells were observed in tissue sections of animals treated with the implantable material at a rate of 6.9±1.2% (P<0.05) in the adventitia; 2.3±0.4% (P<0.05) in the media; and 0.8±0.2% (P<0.05) in the intima. MMP-2 expression remained relatively unchanged from 3 days to one month in animals treated with the implantable material.

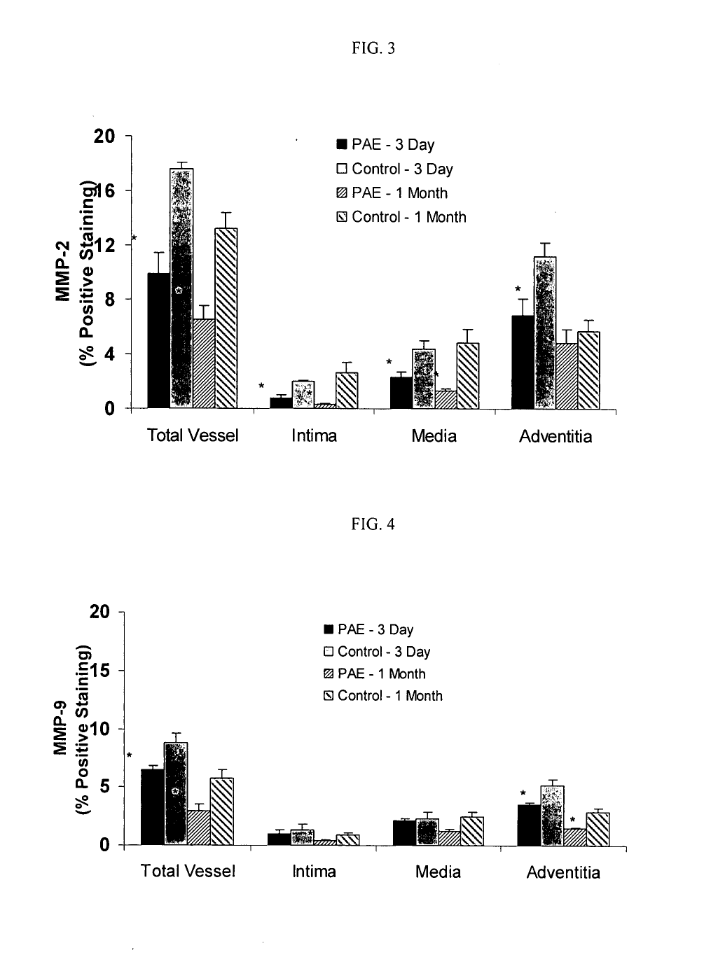

FIG. 3 is a graphical representation of the expression of MMP-2 in stained tissue sections of subjects treated with the implantable material and subjects administered the control material at 3 days and at 1 month. A significant decrease in expression of MMP-2 in veins treated with the implantable material compared to veins administered the control material is evident. Decreased MMP-2 expression was observed in the intima, media and adventitia of veins treated with the implantable material at 3 days and at 1 month.

MMP-9 expression was less intense at both time points for both animals receiving the control material and animals receiving the implantable material compared to MMP-2 expression, discussed above. At day 3, there was reduced staining in the adventitia of veins treated with the implantable material compared to control. At 1 month, decreased MMP-9 expression was observed in the intima and adventitia of the treated group compared to the control (P<0.05). FIG. 4 is a graphical representation of the expression of MMP-9 in stained tissue sections of subjects treated with the implantable material and subjects administered the control material at 3 days and at 1 month.

Wishing not to be bound by theory, it is believed that the implantable material of the present invention restores the proteolytic balance, or the balance between MMPs and TIMPs, in structures treated with the implantable material. Tissue structures constitutively secrete MMPs and TIMPs in a very tightly controlled ratio. However, injury or disease of a tissue structure can induce a deviation in the MMP:TIMP ratio in the structure sufficient to initiate a cascade of events resulting in abnormal neovascularization. The implantable material decreases expression of MMPs or increases expression of TIMPs to restore the balance between MMPs and TIMPs sufficient to reduce abnormal neovascularization or restore normal neovascularization to the treated structure.

### 2. Resection of Colon Tumors in Nude Mice

These studies exemplify use of the present invention's materials and methods to modulate pathological angiogenesis, abnormal neovascularization, extracellular matrix degradation and expression levels of MMPs and TIMPs following tumor resection surgery. Further, the examples, when read in conjunction with the teachings in the detailed description above, provide experimental protocols for testing and using a preferred embodiment of the present invention to reduce or modulate indicia of extracellular matrix degradation, pathological angiogenesis, abnormal neovascularization and expression levels of MMPs and TIMPs following an intervention to resect a solid tumor in an animal subject.

According to this example, colon cancer cells are injected into a cavity on the back of nude mice and allowed to grow into tumors weighing 0.5-1.0 g. The tumors are surgically resected and the resection cavity surgically closed. Two groups of mice will be maintained similarly, except that the treatment group will receive an effective amount of the implantable material to the resection cavity prior to surgical closure. Reduction and/or amelioration of pathological angiogenesis, extracellular matrix degradation, expression levels of MMPs and indicia of inflammation will be monitored over time by laparoscopy, ultrasound, MRI or by sacrificing the mice and examining the resection cavity microscopically. It is expected that mice treated with the implantable material of the present invention will display reduction and/or amelioration of pathological angiogenesis, abnormal neovascularization, extracellular matrix degradation, expression levels of MMPs and/or indicia of inflammation compared to the control mice.

### 3. Resection of Lung Tumors in Lewis Lung Carcinoma Mice

According to this example, Lewis lung carcinoma cells are injected into a cavity on the back of mice according to the mouse model described by O′Reilly et al. in Cell 88:277-285 (1997). The carcinoma cells are allowed to grow into primary tumors weighing 0.5-1.0 g. The primary tumors are surgically resected and the resection cavity surgically closed. Two groups of mice will be maintained similarly, except that the treatment group will receive an effective amount of the implantable material to the resection cavity prior to surgical closure.

Reduction and/or amelioration of pathological angiogenesis, abnormal neovascularization, extracellular matrix degradation, expression levels of MMPs and indicia of inflammation will be monitored over time by laparoscopy, ultrasound, MRI or by sacrificing the mice and examining the resection cavity microscopically. Further, inhibition of regrowth of cancer cells at the site of the resected primary tumor and inhibition of cancer cell metastasis to the lung will be monitored over time by laparoscopy, ultrasound, MRI or by sacrificing the mice and examining the resection cavity and lung microscopically. It is expected that mice treated with the implantable material of the present invention will display reduction and/or amelioration of pathological angiogenesis, abnormal neovascularization, extracellular matrix degradation, expression levels of MMPs, indicia of inflammation, regrowth of cancer cells at the resection cavity and/or metastasis of the cancer cells to the resection margin, to the lung or to surrounding tissue compared to the control mice.

### 4. Resection of Tumors in Humans

Human patients that have been diagnosed with a solid tumor will be studied to demonstrate treatment or management of resection sites following tumor resection. Patients will be examined to identify a solid tumor. Two groups of patients will be maintained similarly, except one group will receive an effective amount of the implantable material to the resection cavity following excision of the solid tumor. The implantable material will be applied at or adjacent to non-cancer cells and/or their stroma at the resection cavity. Reduction and/or amelioration of pathological angiogenesis, abnormal neovascularization, extracellular matrix degradation, expression levels of MMPs and indicia of inflammation will be monitored over time by ultrasound, MRI, CT scan, physical exam, and other relevant procedures depending on the type of resection cavity present in the patient. It is expected that patients treated with the implantable material will display reduction and/or amelioration of pathological angiogenesis, abnormal neovascularizaton, extracellular matrix degradation, expression levels of MMPs and/or indicia of inflammation at the resection cavity and at surrounding tissues compared to the control patients.

### 5. Chemotherapy and/or Radiation Therapy in Humans

Human patients that have been diagnosed with a tumorous growth will be studied to demonstrate treatment or management of the tumor site following chemotherapy and/or radiation therapy at the tumor site to resolve the tumor. Patients will be examined to identify a tumorous growth suitable for treatment with chemotherapy and/or radiation therapy. Two groups of patients will be maintained similarly, except one group will receive an effective amount of the implantable material to the treated tumor site following administration of chemotherapy and/or radiation therapy.

Reduction and/or amelioration of pathological angiogenesis, abnormal neovascularization, extracellular matrix degradation, expression levels of MMPs and indicia of inflammation will be monitored over time by ultrasound, MRI, CT scan, physical exam, and other relevant procedures depending on the type of treatment site present in the patient. It is expected that patients treated with the implantable material will display reduction and/or amelioration of pathological angiogenesis, abnormal neovascularization, extracellular matrix degradation, expression levels of MMPs and/or indicia of inflammation at the treatment site and at surrounding tissues compared to the control patients.

### 6. Treatment of Macular Degeneration in Mice

According to this example, mice with ischemia-induced retinal neovascularization will be studied to demonstrate treatment or management of the eye to reduce the symptoms of and/or delay the onset of macular degeneration according to the model described by Hangai et al. in Am. J. Pathology 161:1429-1437 (2002). Two groups of mice will be maintained similarly, except one group will receive an effective amount of the implantable material to the treated eye or surrounding tissues.

Reduction and/or amelioration of abnormal or pathological neovascularization, expression levels of MMPs and indicia of inflammation will be monitored over time by laparoscopy, ultrasound, MRI or by sacrificing the mice and examining the eye microscopically. It is expected that mice treated with the implantable material of the present invention will display reduction and/or amelioration of abnormal or pathological neovascularization, expression levels of MMPs and indicia of inflammation at the affected tissue compared to the control mice.

### 7. Rheumatoid Arthritis in Mice

According to this example, mice with rheumatoid arthritis will be studied to demonstrate treatment or management of the arthritic joint to reduce the symptoms of and/or delay the onset of rheumatoid arthritis according to the model described by Li et al. in PNAS 103:17432-17437 (2006). Two groups of mice will be maintained similarly, except one group will receive an effective amount of the implantable material to the treated joint or surrounding tissues.

Reduction and/or amelioration of abnormal or pathological neovascularization, expression levels of MMPs and indicia of inflammation will be monitored over time by laparoscopy, ultrasound, MRI or by sacrificing the mice and examining the joint microscopically. It is expected that mice treated with the implantable material of the present invention will display reduction and/or amelioration of abnormal or pathological neovascularization, expression levels of MMPs and indicia of inflammation at the affected tissue compared to the control mice.

### 8. Rheumatoid Arthritis in Humans

Human patients that have been diagnosed with rheumatoid arthritis will be studied to demonstrate treatment or management of the affected joint to reduce the symptoms of and/or delay the onset of rheumatoid arthritis. Patients will be examined to identify a joint suitable for treatment. Two groups of patients will be maintained similarly, except one group will receive an effective amount of the implantable material to the treated joint or surrounding tissues.

Reduction and/or amelioration of extracellular matrix degradation, abnormal neovascularization, expression levels of MMPs and indicia of inflammation will be monitored over time by laparoscopy, ultrasound, MRI or by sacrificing the mice and examining the joint and surrounding tissues microscopically. It is expected that mice treated with the implantable material of the present invention will display reduction and/or amelioration of abnormal neovascularization, extracellular matrix degradation, expression levels of MMPs and indicia of inflammation at the affected joint and/or surrounding tissues compared to the control mice.

### 9. Psoriatic Arthritis in Mice

Mice that have been diagnosed with psoriatic arthritis will be studied to demonstrate treatment or management of the joint to reduce the symptoms of and/or delay the onset of psoriatic arthritis. Two groups of mice will be maintained similarly, except one group will receive an effective amount of the implantable material to the treated joint or surrounding tissues.

Reduction and/or amelioration of abnormal neovascularization, extracellular matrix degradation, expression levels of MMPs and indicia of inflammation will be monitored over time by laparoscopy, ultrasound, MRI or by sacrificing the mice and examining the joint and surrounding tissues microscopically. It is expected that mice treated with the implantable material of the present invention will display reduction and/or amelioration of abnormal neovascularization, extracellular matrix degradation, expression levels of MMPs and indicia of inflammation at the affected joint and/or surrounding tissues compared to the control mice.

### 10. Psoriasis in Mice

Mice that have been diagnosed with psoriasis will be studied to demonstrate treatment or management of the skin to reduce the symptoms of and/or delay the onset of psoriasis. Two groups of mice will be maintained similarly, except one group will receive an effective amount of the implantable material to the treated psoriatic lesion or surrounding tissues.

Reduction and/or amelioration of extracellular matrix degradation, abnormal neovascularization, expression levels of MMPs and indicia of inflammation will be monitored over time by laparoscopy, ultrasound, MRI or by sacrificing the mice and examining the skin microscopically. It is expected that mice treated with the implantable material of the present invention will display reduction and/or amelioration of abnormal neovascularization, extracellular matrix degradation, expression levels of MMPs and indicia of inflammation at the psoriasis lesion compared to the control mice.

### 11. Angiogenesis in an Ex-Vivo Rat Aortic Ring Model

According to this example, the ex-vivo rat aortic ring model of angiogenesis will be studied to demonstrate the ability of the implantable material to regulate angiogenesis according to the model described by Kruger et al. in Biochem. Biophy. Research Comm. 268:183-191 (2000). Thoracic aortas will be excised from eight to ten week old male Sprague-Dawley rats and the fibroadipose tissue removed. The aortas will be sectioned into 1 mm long cross sections, rinsed and placed on Matrigel coated wells. The rat aortic ring sections will be maintained similarly, except one group will receive an effective amount of the implantable material, one group will receive an effective amount of conditioned media, and one group will receive control media.

Reduction and/or amelioration of extracellular matrix degradation, abnormal neovascularization, expression levels of MMPs and indicia of inflammation will be monitored over time by examining the aortic ring sections microscopically. It is expected that rat aortic rings treated with the implantable material of the present invention will display reduction and/or amelioration of abnormal neovascularization, extracellular matrix degradation, expression levels of MMPs and indicia of inflammation compared to the control.

### 12. Matrigel Plug Assay of Angiogenesis in Mice

According to this example, the mouse Matrigel plug assay for angiogenesis will be studied to demonstrate ability of the implantable material to regulate angiogenesis according to the model described by Chander et al. in British J. Cancer 96:1368-1376 (2007). Two groups of mice will be maintained similarly, except one group will receive an effective amount of the implantable material to the Matrigel plug.

Reduction and/or amelioration of abnormal or pathological angiogenesis, expression levels of MMPs and indicia of inflammation will be monitored over time by laparoscopy, ultrasound, MRI or by extracting the Matrigel plug and examining the sections microscopically. It is expected that Matrigel plugs treated with the implantable material of the present invention will display reduction and/or amelioration of abnormal or pathological angiogenesis, expression levels of MMPs and indicia of inflammation at the affected tissue compared to the control mice.

### 13. Corneal Pocket Assay of Corneal Neovascularization in Mice

According to this example, the mouse corneal pocket assay for corneal neovascularization will be studied to demonstrate ability of the implantable material to regulate neovascularization according to the model described by Cao, R. et al. Cancer Cell 6: 333-345 (2004). Briefly, a pellet of Sucrafate (Sigma-Aldrich, St. Louis, Mo.) and hydron [poly(2-HEMA)] (Sigma-Aldrich, St. Louis, Mo.) containing human recombinant bFGF and VEGF will be implanted into each corneal pocket created surgically in 6- to 7-week-old mice. The pellet will be positioned 1.0-1.4 mm from the corneal limbal vessel. After implantation, antibiotic ophthalmic ointment will be applied to each eye. Two groups of mice will be maintained similarly, except one group will receive an effective amount of the implantable material subcutaneously or intraperitoneally.

To induce neovascularization, the cornea and limbal epithelia of both eyes will be removed by applying a rotary motion parallel to the limbus. The eyes will be examined by a slit-lamp biomicroscope for measurement of limbal vessel length from the leading edge of the pellet (pellet distance), the longest blood vessel branching upward from the limbus towards the pellet (vessel length), and how far around the circumference of the eye the vessels have grown.

Reduction and/or amelioration of abnormal neovascularization will be monitored over time by examining the sections microscopically. It is expected that mice treated with the implantable material of the present invention will display reduction and/or amelioration of abnormal neovascularization at the affected tissue compared to the control mice.

The invention may be embodied in other specific forms without departing from the spirit or essential characteristics thereof. The present embodiments are therefore to be considered illustrative and not restrictive, the scope of the invention being indicated by the appended claims rather than by the foregoing description, and all changes which come within the meaning and range of equivalency of the claims are therefore intended to be embraced therein.

What is claimed is:

1. A method of treating a site of pathological angiogenesis in an individual in need thereof, the method comprising the step of:
contacting with an implantable material a surface at or adjacent to or in the vicinity of a site of pathological angiogenesis, wherein said implantable material comprises a biocompatible matrix and cells and further wherein said implantable material is in an amount effective to treat the site of pathological angiogenesis in said individual.

2. The method of claim 1 wherein the biocompatible matrix is a flexible planar material.

3. The method of claim 1 wherein the biocompatible matrix is a flowable composition.

4. The method of claim 1 wherein the cells are endothelial, endothelial-like, epithelial, epithelial-like or non-endothelial cells.

5. The method of claim 1 wherein the implantable material regulates extracellular matrix degradation at the site of pathological angiogenesis.

6. The method of claim 1 wherein the implantable material regulates expression of MMPs at the site of pathological angiogenesis.

7. The method of claim 1 wherein the implantable material regulates indicia of inflammation at the site of pathological angiogenesis.

8. The method of claim 1 wherein the site of pathological angiogenesis is a site of tumor resection or radiation therapy.

9. A composition suitable for the treatment or management of a site of pathological angiogenesis, the composition comprising a biocompatible matrix and cells, wherein said composition is in an amount effective to treat or manage the site of pathological angiogenesis.

10. The composition of claim 9 wherein the biocompatible matrix is a flexible planar material.

11. The composition of claim 9 wherein the biocompatible matrix is a flowable composition.

12. The composition of claim 11 wherein the flowable composition further comprises an attachment peptide and the cells are engrafted on or to the attachment peptide.

13. The composition of claim 9 wherein the cells are endothelial, endothelial-like, epithelial, epithelial-like or non-endothelial cells.

14. The composition of claim 9 wherein the composition regulates extracellular matrix degradation at the site of pathological angiogenesis.

15. The composition of claim 9 wherein the composition regulates expression of MMPs at the site of pathological angiogenesis.

16. A method of treating a site of abnormal neovascularization in an individual in need thereof, the method comprising the step of:
contacting with an implantable material a surface at or adjacent to or in the vicinity of a site of abnormal neovascularization, wherein said implantable material comprises a biocompatible matrix and cells and further wherein said implantable material is in an amount effective to treat the site of abnormal neovascularization in said individual.

17. The method of claim 16 wherein the cells are endothelial, endothelial-like, epithelial, epithelial-like or non-endothelial cells.

18. The method of claim 16 wherein the site of abnormal neovascularization is a site of a neovascular disease of the eye selected from the group consisting of macular degeneration, corneal neovascularization, proliferative diabetic retinopathy, retinopathy of prematurity, Steven's-Johnson syndrome, cicatricial pemphigoid and corneal allograft rejection.

19. The method of claim 16 wherein the site of abnormal neovascularization is a site of rheumatoid arthritis or synovial neovascularization.

20. The method of claim 16 wherein the site of abnormal neovascularization is a site of psoriasis or psoriatic arthritis.

21. The method of claim 16 wherein the site of abnormal neovascularization is a site of a systemic inflammatory disease.

22. A composition suitable for the treatment or management of a site of abnormal neovascularization, the composition comprising a biocompatible matrix and cells, wherein said composition is in an amount effective to treat or manage the site of abnormal neovascularization.

23. The composition of claim 22 wherein the biocompatible matrix is a flexible planar material.

24. The composition of claim 22 wherein the biocompatible matrix is a flowable composition.

25. The composition of claim 24 wherein the flowable composition further comprises an attachment peptide and the cells are engrafted on or to the attachment peptide.

26. The composition of claim 22 wherein the cells are endothelial, endothelial-like, epithelial, epithelial-like or non-endothelial cells.

27. The composition of claim 22 wherein the composition regulates extracellular matrix degradation at the site of abnormal neovascularization.

28. The composition of claim 22 wherein the composition regulates expression of MMPs at the site of abnormal neovascularization.
